(19) United States
(12) Patent Application Publication
(10) Pub. No.: US 2023/0037966 A1
(43) Pub. Date: Feb. 9, 2023

(54) SOURCE SPECIFIC EXOSOMES FOR DETERMINING AVOIDANCE OF CANCER TREATMENT AND AVOIDANCE OF CHECKPOINT INHIBITOR THERAPIES

(71) Applicant: The Trustees of the University of Pennsylvania, Philadelphia, PA (US)

(72) Inventors: Wei Guo, Wynnewood, PA (US); Bin Wu, Wynnewood, PA (US)

(21) Appl. No.: 17/790,622

(22) PCT Filed: Jan. 7, 2021
(86) PCT No.: PCT/US2021/012452

§ 371 Date: Jul. 1, 2022

Related U.S. Application Data
(60) Provisional application No. 62/957,966, filed on Jan. 7, 2020.

(51) Int. Cl. C07K 16/30; C07K 16/28; C07K 16/42; G01N 33/50

(52) U.S. Cl. CPC C07K 16/30; C07K 16/2812; C07K 16/42; G01N 33/5076; C07K 2317/24; C07K 2317/76; G01N 2800/52

(57) ABSTRACT

The present disclosure provides methods for predicting and thereby treating cancer or increasing the efficacy of an anti-cancer medication, in part by measuring checkpoint proteins on extracellular vesicles released from non-cancer cells. These checkpoint proteins promote cancer progression and/or compensate for the loss of signal from the checkpoint proteins being inhibited by the checkpoint inhibitory therapy. Compositions and methods of treatment are also provided.

### CROSS-REFERENCE TO RELATED APPLICATION

The present application is entitled to priority under 35 U.S.C. § 119(e) to U.S. Provisional Patent Application No. 62/957,966 filed Jan. 7, 2020, which is hereby incorporated by reference in its entirety herein.

### STATEMENT REGARDING FEDERALLY SPONSORED RESEARCH

This invention was made with government support under R01-GM085146 awarded by the National Institutes of Health. The government has certain rights in this invention.

### BACKGROUND OF THE INVENTION

Checkpoint proteins, such as PD-L1, PD-L2, B7H3, and B7H4, are transmembrane proteins expressed in a variety of cell types including tumor cells. These proteins play a critical role in immunosuppression and/or immunostimulation as they bind to receptors such as PD-1 on activated T cells to elicit immune checkpoint responses. The current model for PD-L1-mediated immunosuppression is centered on the direct interaction between PD-L1 on tumor cell surface and PD-1 on T cells. This interaction delivers a potent inhibitory signal that induces functional “exhaustion” or apoptosis of T cells. PD-L1 on the cancer cell surface can locally inactivate immune cells as an adaptive response to immune pressure. Immune checkpoint blockade-based therapies (i.e. checkpoint inhibitor therapies) have thus been developed to treat many types of cancers. For example, blocking antibodies that target the PD-L1/PD-1 axis have shown remarkable promise in the treatment of number of solid tumors, including metastatic melanoma. However, only a minority of patients respond to the therapy and the responses are often partial or short-lived. For example, the percentage of patients estimated to respond to checkpoint inhibitor therapies was just 12.46% in 2018.

One theory for the high non-responsive rate is that cancer cells release checkpoint proteins that are bound to extracellular vesicles. PD-L1 proteins have been identified in the blood samples of patients with melanoma, renal cell carcinoma, hepatocellular carcinoma, multiple myeloma, and B-cell lymphoma; the level of blood PD-L1 proteins appears to correlate positively with tumor progression and negatively with patients' overall survival. It is believed that as tumors get larger, they release more checkpoint proteins overwhelming the potential therapy. Nonetheless, these theories have not led to significant increases in the response rate to checkpoint inhibitor therapies and fail to account for why patient responses are often partial or short-lived, even when tumor masses decrease.

There is a need in the art for better molecular understanding of the interplay between tumor cells, host somatic cells, and the immune system in order to improve current therapies and to identify patients most likely to respond to a first checkpoint inhibitor therapy. There is also a need to provide alternative checkpoint inhibitor therapies or to increase the likelihood of success for the first checkpoint inhibitor therapy. The present invention addresses these needs.

### SUMMARY OF THE INVENTION

In one aspect, the invention includes a method for treating a patient who fails to respond to a checkpoint inhibitor therapy. The method comprises obtaining a first biological sample from the patient before the administration of the checkpoint inhibitor therapy, and a second biological sample from the patient after the patient has been administered at least one treatment of the checkpoint inhibitor therapy. Each of the first and second biological samples comprises an extracellular vesicle comprising a source-specific marker and a checkpoint protein. The levels of the source-specific marker and checkpoint protein on the extracellular vesicles are assessed from the first and second biological samples. When the amount of the checkpoint protein from the second biological sample is elevated above the amount of the checkpoint protein from the first biological sample, an alternative treatment is administered to the patient.

In certain embodiments, the checkpoint inhibitor therapy comprises an inhibitor of signaling of a first checkpoint protein.

In certain embodiments, the alternative treatment comprises a second checkpoint inhibitor therapy comprising an inhibitor of signaling of a second checkpoint protein.

In certain embodiments, the source-specific marker is selected from the group consisting of a marker for bone marrow derived antigen presenting cells, a marker for stroma cells, a marker for T cells, a marker for B cells, a marker for cancer cells, a marker for liver cells, a marker for stomach cells, a marker for pancreatic cells, a marker for small intestine cells, a marker for large intestine cells, a marker for endocrine cells, a marker for epithelial cells, a marker for endothelial cells, a marker for mesodermal cells, a marker for humoral cells, a marker for bone marrow, a marker for bone cells, a marker for nervous system cells, a marker for brain cells, a marker for neurons, a marker for glial cells, a marker for astrocytes, a marker for microglia, a marker for ependymal cells, a marker for stem cells, a marker for smooth muscle cells, a marker for cardiac cells, a marker for cardiac muscle cells, or a marker for basal cells.

In certain embodiments, the marker for bone marrow derived antigen presenting cells comprises a marker for macrophages. In certain embodiments, the marker for macrophages is selected from the group consisting of a marker for M2 macrophages, a marker for M1 macrophages, a marker for tumor-associated macrophages, CD163, CD115, or CD11b. In certain embodiments, the bone marrow derived antigen presenting cells comprises M2 macrophages or tumor-associated macrophages.

In certain embodiments, the marker for T cells comprises a marker for CD8+ T cells, a marker for CD4+ T cells, or a marker for regulatory T cells.

In certain embodiments, the marker for stromal cells comprises CD44, CD90, CD105, or Fibroblast Surface Antigen (SFA).

In certain embodiments, the first checkpoint inhibitor therapy comprises an inhibitor of programmed cell death protein 1 (PD-1) or programmed death-ligand protein 1 (PD-L1). In certain embodiments, the inhibitor of PD-L1 comprises atezolizumab.

In certain embodiments, the source-specific marker is selected from the group consisting of CD63, CD9, C81, CD163, CD11b, CD11c, CD115, Gr-1, CD8, CD4, CD44, CD90, CD105, SFA, epidermal growth factor receptor (EGFR), vascular endothelial growth factor receptor (VEGFR), PD-L1, CD155, CD112, B7-1, B7-2, B7-H, Galectin-9, Siglec-15, OX40 receptor ligand (OX40L), major histocompatibility complex class II molecules (MHC-II), ICAM-1, LFA-1, B7-H2, CD40, CD70, CD48, CD52, CD137, TL1A, GITRL, CD30L, T cell immunoglobin and mucin domain 1 (TIM1), TIM3, TIM4, signaling lymphocytic activation molecule (SLAM), tumor necrosis factor superfamily member 14 (TNFSF14 or LIGHT), herpesvirus entry mediator (HVEM), PD-1, cytotoxic T lymphocyte-associated protein (CTLA-4), T cell immunoreceptor with Ig and immunoreceptor tyrosine-based inhibitory motif domains (TIGIT), Poliovirus Receptor Related Immunoglobulin Domain Containing (PVRIG), lymphocyte-activation gene 3 (LAG3), OX40, V-set and immunoglobulin domain containing 3 (VISG3), VISG8, inducible T cell costimulator (ICOS), CD28, CD40L, death receptor 3 (DR3), glucocorticoid-induced tumor necrosis factor-related protein (GITR), CD30, CD2, CD226, CD160, B and T lymphocyte attenuator (BTLA), programed death-1 homolog (PD-1H), leukocyte-associated immunoglobulin-like receptor 1 (LAIR1), or natural killer cell receptor 2B4 (NKCR2B4).

In certain embodiments, the second checkpoint protein comprises: PD-L1, CD155, CD112, B7-1, B7-2, B7-H3, Galectin-9, Siglec-15, MHC-II, B7-H2, CD70, CD48, CD52, CD137, TL1A, GITRL, CD30L, TIM1, TIM3, TIM4, SLAM, LIGHT, HVEM, PD-1, CTLA-4, TIGIT, PVRIG, LAG3, VISG-3, VISG-8, ICOS, CD28, DR3, GITR, CD30, CD2, CD226, CD160, BTLA, PD1H, LAIR1, or NKCR2B4.

In certain embodiments, the second checkpoint protein and the source-specific marker are the same. In certain embodiments, the second checkpoint protein and the source-specific marker differ.

In certain embodiments, the second checkpoint protein is a receptor and the inhibitor of the signaling of the second checkpoint protein is an inhibitor of a ligand to the receptor.

In certain embodiments, the second checkpoint protein is a ligand and the inhibitor of the signaling of the second checkpoint protein is an inhibitor of a receptor to the ligand. In certain embodiments, the second checkpoint protein is a ligand and a receptor.

In certain embodiments, the second checkpoint protein is PD-1.

In certain embodiments, the second checkpoint protein is CD155 and the inhibitor therapy is a TIGIT inhibitor.

In certain embodiments, the difference in total amounts of extracellular vesicles between the first and second biological samples are normalized. In certain embodiments, the normalizing comprises measuring in each of the first and second biological samples: extracellular vesicles comprising CD9, extracellular vesicles comprising CD63, and extracellular vesicles comprising CD81, thereby measuring the total amount of extracellular vesicles in each of the first and second biological samples. In certain embodiments, in the normalizing, a total amount of the second checkpoint protein in the total amount of extracellular vesicles is obtained for each of the first and second biological samples.

In certain embodiments, the administering is when the amount of the second checkpoint protein on the extracellular vesicle that has the source-specific marker in the second biological sample per the total amount of the second checkpoint protein in the total amount of extracellular vesicles in the second biological sample is elevated above the amount of the second checkpoint protein on the extracellular vesicle that has the source-specific marker of the first biological sample per the total amount of the second checkpoint protein in the total amount of extracellular vesicles in the first biological sample.

In certain embodiments, the amount of the checkpoint protein on the extracellular vesicle that has the source-specific marker of each of the first and second biological samples is obtained by binding the checkpoint protein to a labelled antibody.

In certain embodiments, the extracellular vesicle that has a source-specific marker is not from a cancer cell. In certain embodiments, the source-specific marker excludes a marker of a cancer cell.

In certain embodiments, the alternative treatment is administered when the amount of the checkpoint protein from the second biological sample is elevated at least 1.5, 2, 3, 4, 5, 6, 7, 8, 9, 10, or 20 fold above the amount of the checkpoint protein from the first biological sample.

In another aspect, the invention includes a method of identifying whether a subject is responsive to a checkpoint protein inhibitor therapy. The method comprises i) detecting the level of an extracellular vesicle-bound immune regulatory protein obtained from the subject; ii) detecting the level of a marker for the extracellular vesicle; and iii) identifying the subject as non-responsive to treatment. The subject is identified as non-responsive to treatment when: a) the level of the extracellular vesicle-bound immune regulatory protein and the level of the marker for the extracellular vesicle correlate in a population of individuals, and b) there is a X increase in the level of the extracellular vesicle-bound immune regulatory protein from the subject per the level of marker for the extracellular vesicle from the subject above Y. Y equals the mean of Z, and X is the standard deviation of Z. Z is levels of the extracellular vesicle-bound immune regulatory protein of each individual from the population of individuals divided by levels of the marker for the extracellular vesicle of said each individual from the population of individuals, wherein X is at least 0.68.

In certain embodiments, the marker for the extracellular vesicle comprises: CD63, CD9, C81, CD163, CD11b, CD11c, CD115, Gr-1, CD8, CD4, CD44, CD90, CD105, SFA, epidermal growth factor receptor (EGFR), vascular endothelial growth factor receptor (VEGFR), PD-L1, CD155, CD112, B7-1, B7-2, B7-H, Galectin-9, Siglec-15, OX40 receptor ligand (OX40L), major histocompatibility complex class II molecules (MHC-II), ICAM-1, LFA-1, B7-H2, CD40, CD70, CD48, CD52, CD137, TL1A, GITRL, CD30L, T cell immunoglobin and mucin domain 1 (TIM1), TIM3, TIM4, signaling lymphocytic activation molecule (SLAM), tumor necrosis factor superfamily member 14 (TNFSF14 or LIGHT), herpesvirus entry mediator (HVEM), PD-1, cytotoxic T lymphocyte-associated protein (CTLA-4), T cell immunoreceptor with Ig and immunoreceptor tyrosine-based inhibitory motif domains (TIGIT), Poliovirus Receptor Related Immunoglobulin Domain Containing (PVRIG), lymphocyte-activation gene 3 (LAG3), OX40, V-set and immunoglobulin domain containing 3 (VISG3), VISG8, inducible T cell costimulator (ICOS), CD28, CD40L, death receptor 3 (DR3), glucocorticoid-induced tumor necrosis factor-related protein (GITR), CD30, CD2, CD226, CD160, B and T lymphocyte attenuator (BTLA), programed death-1 homolog (PD-1H), leukocyte-associated immunoglobulin-like receptor 1 (LAIR1), or natural killer cell receptor 2B4 (NKCR2B4).

In certain embodiments, the extracellular vesicle-bound immune regulatory protein comprises: PD-L1, CD155, CD112, B7-1, B7-2, B7-H3, Galectin-9, Siglec-15, OX40L, MHC-II, B7-H2, CD40, CD70, CD48, CD52, CD137, TL1A, GITRL, CD30L, TIM1, TIM3, TIM4, SLAM, LIGHT, HVEM, PD-1, CTLA-4, TIGIT, PVRIG, LAG3, OX40, VISG-3, VISG-8, ICOS, CD28, CD40L, DR3, GITR, CD30, CD2, CD226, CD160, BTLA, PD1H, LAIR1, or NKCR2B4.

In certain embodiments, X is at least 1, at least 1.5, at least 2, at least 2.5, or at least 3.

In another aspect, the invention includes a method of increasing the efficacy of an anti-cancer medication in a patient in need thereof. The method comprises contacting a biological sample from the patient with a first reagent. The biological sample comprises a cancer-promoting extracellular vesicle comprising a checkpoint protein and a source-specific marker. The checkpoint protein suppresses an immune response to the cancer. In the contacting, the first reagent binds the cancer-promoting extracellular vesicles, thereby removing the cancer-promoting extracellular vesicles from the biological sample. A purified biological sample is obtained and introduced into the patient, thereby increasing the efficacy of the anti-cancer medication.

In certain embodiments, the first reagent binds the source-specific marker, thereby removing the cancer-promoting extracellular vesicles from the biological sample.

In certain embodiments, the anti-cancer medication comprises a checkpoint inhibitor therapy comprising an inhibitor of signaling of the checkpoint protein.

In certain embodiments, the source-specific marker is selected from the group consisting of a marker for bone marrow derived antigen presenting cells, a marker for stroma cells, a marker for T cells, a marker for B cells, a marker for cancer cells, a marker for liver cells, a marker for stomach cells, a marker for pancreatic cells, a marker for small intestine cells, a marker for large intestine cells, a marker for endocrine cells, a marker for epithelial cells, a marker for endothelial cells, a marker for mesodermal cells, a marker for humoral cells, a marker for bone marrow, a marker for bone cells, a marker for nervous system cells, a marker for brain cells, a marker for neurons, a marker for glial cells, a marker for astrocytes, a marker for microglia, a marker for ependymal cells, a marker for stem cells, a marker for smooth muscle cells, a marker for cardiac cells, a marker for cardiac muscle cells, or a marker for basal cells.

In certain embodiments, the marker for bone marrow derived antigen presenting cells comprises a marker for macrophages. In certain embodiments, the marker for macrophages comprises a marker for M2 macrophages, M1 macrophages, or tumor-associated macrophages. In certain embodiments, the marker for macrophages comprises CD63, CD115, or CD11b. In certain embodiments, the bone marrow derived antigen presenting cells comprises M2 macrophages or tumor-associated macrophages.

In certain embodiments, the marker for T cells comprises a marker for CD8+ T cells, a marker for CD4+ T cells, or a marker for regulatory T cells.

In certain embodiments, the marker for stromal cells comprises CD44, CD90, CD105, or Fibroblast Surface Antigen (SFA).

In certain embodiments, the checkpoint inhibitor therapy comprises an inhibitor of programmed cell death protein 1 (PD-1) or an inhibitor of programmed death-ligand protein 1 (PD-L1). In certain embodiments, the inhibitor of PD-L1 comprises atezolizumab.

In certain embodiments, the source-specific marker comprises: CD63, CD9, C81, CD163, CD11b, CD11c, CD115, Gr-1, CD8, CD4, CD44, CD90, CD105, SFA, epidermal growth factor receptor (EGFR), vascular endothelial growth factor receptor (VEGFR), PD-L1, CD155, CD112, B7-1, B7-2, B7-H, Galectin-9, Siglec-15, OX40 receptor ligand (OX40L), major histocompatibility complex class II molecules (MHC-II), ICAM-1, LFA-1, B7-H2, CD40, CD70, CD48, CD52, CD137, TL1A, GITRL, CD30L, T cell immunoglobin and mucin domain 1 (TIM1), TIM3, TIM4, signaling lymphocytic activation molecule (SLAM), tumor necrosis factor superfamily member 14 (TNFSF14 or LIGHT), herpesvirus entry mediator (HVEM), PD-1, cytotoxic T lymphocyte-associated protein (CTLA-4), T cell immunoreceptor with Ig and immunoreceptor tyrosine-based inhibitory motif domains (TIGIT), Poliovirus Receptor Related Immunoglobulin Domain Containing (PVRIG), lymphocyte-activation gene 3 (LAG3), OX40, V-set and immunoglobulin domain containing 3 (VISG3), VISG8, inducible T cell costimulator (ICOS), CD28, CD40L, death receptor 3 (DR3), glucocorticoid-induced tumor necrosis factor-related protein (GITR), CD30, CD2, CD226, CD160, B and T lymphocyte attenuator (BTLA), programed death-1 homolog (PD-1H), leukocyte-associated immunoglobulin-like receptor 1 (LAIR1), or natural killer cell receptor 2B4 (NKCR2B4).

In certain embodiments, the checkpoint protein comprises: PD-L1, CD155, CD112, B7-1, B7-2, B7-H3, Galectin-9, Siglec-15, B7-H2, CD70, CD48, CD52, CD137, TL1A, GITRL, CD30L, TIM1, TIM3, TIM4, SLAM, LIGHT, HVEM, PD-1, CTLA-4, TIGIT, PVRIG, LAG3, OX40, VISG-3, VISG-8, ICOS, CD28, DR3, GITR, CD30, CD2, CD226, CD160, BTLA, PD1H, LAIR1, or NKCR2B4.

In certain embodiments, the checkpoint protein and the source-specific marker are the same. In certain embodiments, the checkpoint protein and the source-specific marker differ.

In another aspect, the invention includes a method for treating cancer in a patient in need thereof. The method comprises assessing the levels of PD-L1 and/or PD-1 and the levels of CD155 and/or CD112 and/or TIGIT in a biological sample comprising an extracellular vesicle obtained from the patient before administration of a first therapy. When the the level of PD-L1 and/or PD-1 is high and the level of CD155 and/or CD112 and/or TIGIT is low in comparison to a reference sample, the first therapy is administered to the patient. When the levels of PD-L1 and/or PD-1 and the levels of CD155 and/or CD112 and/or TIGIT are high in comparison to a reference sample, a second therapy is administered alone or together with the first therapy to the patient.

In certain embodiments, the first therapy comprises an inhibitor of PD-1 or PD-L1 and the second therapy comprises an inhibitor of TGIT. In certain embodiments, the inhibitor is selected from the group consisting of an antibody, a chemical compound, or an siRNA.

In another aspect, the invention includes a method for treating cancer in a patient in need thereof. The method comprises assessing the level of CD155 and/or CD112 in a biological sample comprising an extracellular vesicle obtained from the patient before administration of a first therapy, and assessing the level of CD155 and/or CD112 in a biological sample comprising an extracellular vesicle obtained from the patient after administration of a first therapy. When the the level of CD155 and/or CD112 in the second biological sample is increased in comparison to the first biological sample, the patient is determined to not be responding to the first therapy, and a second therapy is administered to the patient.

In certain embodiments, the first therapy comprises an inhibitor of PD-1 or PD-L1 and the second therapy comprises an inhibitor of TGIT.

In certain embodiments, the inhibitor is selected from the group consisting of an antibody, a chemical compound, or an siRNA.

In another aspect, the invention includes a method for treating cancer in a patient in need thereof. The method comprises assessing the level of a co-stimulatory factor in a first biological sample comprising an extracellular vesicle obtained from the patient before administration of a therapy, and assessing the level of the co-stimulatory factor in a second biological sample comprising an extracellular vesicle obtained from the patient after administration of a therapy.

When the level of the co-stimulatory factor is increased in the second biological sample in comparison to the first biological sample, the patient is determined to be responding to therapy, and the therapy is continued.

In certain embodiments, the method further comprises assessing the level of the co-stimulatory factor in a third biological sample comprising an extracellular vesicle obtained from the patient. When the level of the co-stimulatory factor is decreased in the third biological sample in comparison to the first biological sample, the patient is determined to be responding to therapy, and the therapy is continued. When the level of the co-stimulatory factor is increased in the third biological sample in comparison to the first biological sample, the patient is determined to not be responding to therapy, and the therapy is discontinued and a alternative therapy is administered to the patient.

In certain embodiments, the second biological sample is obtained three weeks after the first biological sample. In certain embodiments, the third biological sample is obtained 2, 3, 4, 5, 6, 7, 8, or 9 weeks after the second biological sample.

In certain embodiments, the co-stimulatory factor is selected from the group consisting of CD40, CD40L, OX40, OX40L, CD137, and CD137L.

In another aspect, the invention includes a method for treating cancer in a patient in need thereof. The method comprises assessing the level of phosphorylated HRS in a biological sample comprising tumor tissues or an extracellular vesicle obtained from the patient. When the level of phosphorylated HRS is high in comparison to a reference sample, the patient is administered a combination therapy.

In certain embodiments, the combination therapy comprises a drug that inhibits PD-1 or PD-L1 and a drug that inhibits the MAPK pathway.

In certain embodiments, the cancer is melanoma.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1K depict various experimental methods and results demonstrating detection, capture, and measurements of exosomes carrying source-specific or source-nonspecific markers, and checkpoint proteins. FIG. 1A: Immunoblotting detection of checkpoint immunosuppressive proteins (e.g. CD155, CD112, CD113, Galectin 9, PD-L1, B7-H3), immunostimulatory proteins (e.g. OX40L), and source-specific proteins (e.g. EGFR, CD9), and other exosome proteins such as GAPDH and MHC-I in exosomes (“EXO”) and whole cell lysates (“WCL”) derived from pancreatic cancer cells lines PANC-1 and Mia-PaCa2. FIG. 1B: A representative TEM image of exosomes purified from the culture supernatant of human melanoma cells. FIG. 1C: Characterization of the purified exosomes using NANOSIGHT® nanoparticle tracking system. FIG. 1D: Heatmap of reverse phase protein array (RPPA) data showing the levels of PD-L1 in the whole cell lysate (“WCL”) and the exosomes (“EXO”) secreted by primary or metastatic melanoma cell lines (left panel). A graphical representation of Log 2 transformed RPPA results is shown in the right panel. FIG. 1E: Immunoblots for PD-L1 in the whole cell lysate (“W”) and purified exosomes (“E”). The same amounts of whole cell lysates and exosome proteins were loaded. CD63, Hrs, Alix, and TSG101 were used as exosome markers. GAPDH was used as the loading control. FIG. 1F: Density gradient centrifugation confirming that PD-L1 secreted by metastatic melanoma cells co-fractionated with exosome markers CD63, Hrs, Alix, and TSG101. FIG. 1G: Diagram of enzyme-linked immunosorbent assay (“ELISA”) of exosomal PD-L1 using monoclonal antibodies against the extracellular domain of PD-L1. FIG. 1H: PD-L1 on the surface of exosomes secreted by human cells as determined by ELISA. FIG. 11: A representative TEM image of melanoma cell-derived exosomes immunogold-labeled with a monoclonal antibody against the extracellular domain of PD-L1. Arrowheads indicate 5-nm gold particles. FIG. 1J: Diagram of flow cytometric analysis of exosomal PD-L1 by CD63-coated beads. FIG. 1K: Secretion of PD-L1 protein on exosome surface by human melanoma cells as determined by flow cytometry, indicating the percentage of beads with PD-L1 exosomes from a representative experiment.

FIG. 2 depicts results measuring immunomodulators including checkpoint proteins on total exosomes. Total exosomes derived from the Panc-1 pancreatic cancer cell line were captured by combination of antibodies against CD63, CD9, and CD81. Biotin labeled anti-CD63, anti-B7-H3, and anti-CD155 antibodies were used to detect the proteins on exosomes.

FIG. 3 depicts the results of an ELISA detecting CD155 protein obtained from exosomes derived from macrophages, and in particular exosomes captured with an anti-CD163 antibody, from melanoma patients. Without further analysis weighing the contributions of the CD163 positive exosomes derived from macrophages from the patients, it is difficult to predict whether an individual patient will respond to a checkpoint inhibitor therapy, such as an anti-PD-1 antibody. Alternatively, without further temporal information, such as CD155 levels on CD163 positive exosomes, before and after treatment with a checkpoint inhibitor therapy, it is difficult to predict whether an individual patient will respond to the therapy.

FIG. 4 depicts the results of an enzyme-linked immunosorbent assay (“ELISA”) detecting CD155 protein obtained from exosomes captured with an anti-PD-L1 antibody from melanoma patients. Without further analysis of the correlation of PD-L1 and CD155 or other checkpoint proteins on the circulating exosomes, it is difficult to predict whether an individual patient will respond to a checkpoint inhibitor therapy, such as an anti-PD-1 antibody.

FIG. 5 depicts results demonstrating that patients who failed to respond to PD-1 inhibitor therapy had a significant increase in CD155 protein levels on circulating CD11b positive exosomes during week 9 of an anti-PD-1 antibody therapy over that of week 6 than that of patients who responded to the therapy (t-test, p=0.0229). CD155 protein levels on circulating exosomes captured with an anti-CD11b antibody were quantified in 8 patients who responded and 9 patients who did not respond to PD-1 inhibitor therapy. Samples were taken throughout the course of the patients' 9 week therapy. The CD155 protein levels on CD11b-positive exosomes from week 9 were divided by the CD155 protein levels on CD11b-positive exosomes from week 6.

FIG. 6 depicts results quantifying CD155 protein levels on circulating exosomes captured with an anti-CD11b antibody in patients who responded and who did not respond to PD-1 inhibitor therapy throughout the 9-week therapy (week 0 being before treatment). The patients who failed to respond to PD-1 inhibitor therapy also had significant increases in CD155 protein on circulating CD11b positive exosomes at around 6 and 9 weeks treatment than that of patients who responded to therapy (arrows). Without wishing to be bound by a particular theory, it is believed that because CD155 is an immunosuppressive checkpoint protein, elevations in CD155 on CD11b-positive exosomes or macrophage exosomes during an anti-cancer therapy may cause short-lived responses and/or non-responses to therapy because at least CD155 may serve to bypass the loss of signaling of another checkpoint protein by the checkpoint inhibitor therapy.

FIG. 7 depicts the correlation of the levels of total exosomes and the levels of PD-L1 on CD163-positive exosomes in patients who respond to PD-1 inhibitor therapy (left), whereas there was no correlation in patients who failed to respond to PD-1 inhibitor therapy (middle). It can thus be predicted whether a patient will fail to respond to PD-1 inhibitor therapy, other checkpoint therapies, or other anti-cancer therapies when that patient has levels of checkpoint protein that significantly deviate from the correlation and regression derived from the patients who responded to said checkpoint inhibitor therapy or said anti-cancer therapy (right). The right graph combines the results from the responders and non-responders and sets a threshold (dotted line) parallel to the regression from the responders (solid line) used to predict whether a patient will respond to a particular therapy.

FIGS. 8A-8D depict diagrams and results demonstrating that the phosphorylation of HRS by ERK can also promote the loading of PD-L1 and other immune regulatory proteins to the exosomes through the HRS protein. FIG. 8A: Phosphorylation site of serine 345 on HRS. FIG. 8B: MAPK pathway activation can trigger HRS phosphorylation. FIG. 8C: Substitution studies of serine 345 with alanine (phosphor-defect) or glutamic acid (phosphor-mimetic) by site-directed mutagenesis. The level of PD-L1 was reduced in exosomes from the cells expressing phosphor-defect mutant HRS S345A; there is more PD-L1 enriched in exosomes from the cells expressing phosphor-mimetic mutant HRS S345D. FIG. 8D: Immunohistochemistry (IHC) analysis of melanoma patient samples shows that CD8 positive T cells failed to infiltrate into the tumor when HRS phosphorylation level in tumor cells is high. Phosphorylated HRS in tumor tissues was detected by a specific rabbit anti-phospho-HRS antibody. High levels of HRS phosphorylation blocks CD8 T cell infiltration and diminishes patient response to checkpoints inhibitors.

FIGS. 9A-9D depict experimental results demonstrating the contribution of Tumor Associated Macrophage (TAM) exosomes to immune suppression in patient tumor tissues using flow cytometry. FIG. 9A: Percent of proliferating T cells labelled with Ki-67 after exposure to total exosomes or exosomes devoid of TAM exosomes (removal with anti-CD163 antibody). FIG. 9B: Percent of cells labelled with granzyme B after exposure to total exosomes or exosomes devoid of TAM exosomes population (removal with anti-CD163 antibody). FIG. 9C: Percent of proliferating cells labelled with granzyme B after exposure to total exosomes from M0 macrophages (“M0”) and tumor associated macrophages (“TAM”). FIG. 9D: Percent of proliferating cells labelled with Ki-67 after exposure to total exosomes from M0 macrophages (“M0”) and tumor associated macrophages (“TAM”).

FIGS. 10A-10D depict experimental results measuring average exosomal PD-L1 levels in responders (FIG. 10A) and non-responders (FIG. 10B), and individual levels of the same (FIG. 10C) across 12 weeks of pembrolizumab treatment, with week 0 being before the first treatment of pembrolizumab, as well as, how CD8 positive T cell reinvigoration (FIG. 10D) precedes PD-L1 levels in responders.

FIGS. 11A-11D illustrate combined analysis of CD155 and PD-L1 on myeloid cell-derived exosmes (CD11b+). FIG. 11A: Analysis of the pre-treatment levels of CD155 and PD-L1 on myeloid cell-derived exosomes (captured with anti-CD11b antibody). FIGS. 11B-11D: Bar plots of RECIST response categories (CR/PR, SD/PD) by PD-L1 and CD155 level on CD11b+ exosomes [CD155High (>0.3) vs. CD155Low (<0.3); PD-L1High (>8.0) vs PD-L1Low(<8.0)] in patients with metastatic melanoma treated with anti-PD-1 monotherapy (n=20 patients).

FIGS. 12A-12C illustrate PD-L1 on Tumor-associated Macrophage (TAM)-derived exosomes (CD163+). FIG. 12A: Comparison of the difference of baseline levels of PD-L1 on TAM-derived exosomes (CD163+) and total PD-L1 in melanoma patients treated with a-PD-1 therapy. Difference (ng/ml)=[PD-L1(CD163+)]−[PD-L1(5H1)]. FIG. 12B: ROC curve analysis for the difference of pretreatment CD163+ exosomal PD-L1 and total exosomal PD-L1 in clinical responders compared to non-responders. FIG. 12C: ORR for patients with high and low difference of exosomal PD-L1 (n=17 patients).

FIGS. 13A-13C illustrate PD-L1 on TAM-derived exosomes (Lung cancer). FIG. 13A: Comparison of the fold change of PD-L1 on TAM-derived exosomes (CD163+) in patients with lung cancer treated with anti-PD-1 antibody. FIG. 13B: ROC curve analysis for the fold change of CD163+ exosomal PD-L1 in clinical responders compared to non-responders. FIG. 13C: ORR for patients with high and low fold change of CD163+ exosomal PD-L1 (n=24 patients).

FIGS. 14A-14B illustrate co-expression of PD-L1 and CD155 in melanoma patient exosomes. FIG. 14A: Comparison of the CD155 levels on PD-L1+ exosomes in melanoma patients compared to health donor. FIG. 14B: Comparison of the PD-L1 levels on exosomes captured by CD155 in melanoma patients compared to health donor.

FIG. 15A: Detection of OX40 in CD8 T cell-derived exosomes (CD8b+ exosomes). FIG. 15B: Detection of CD40 on myeloid cell-derived exosomes (CD11b+ exosomes). FIG. 15C: Detection of OX40L on myeloid cell-derived exosomes (CD11b+ exosomes). FIG. 15D: detection of CD137 on CD8 T cell-derived exosomes (CD8b+ exosomes) or myeloid cell-derived exosomes (CD11b+ exosome).

FIGS. 16A-16C depict CD112 on myeloid cells derived exosomes (CD11b+): correlative and non-correlative expression of CD112 and PD-L1. FIG. 16A: Detection of CD112 in myeloid cell-derived exosomes. FIG. 16B: Detection of total exosomal PD-L1. FIG. 16C: Pearson correlation of the CD11b+ exosomal CD112 and total exosomal PD-L1 in melanoma patients.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Herein, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

Herein the statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition), Michael R. Green and Joseph Sambrook eds., and Harlow et al., Antibodies: A Laboratory Manual, Chapter 14, Cold Spring Harbor Laboratory, Cold Spring Harbor (2013, 2nd edition).

In some embodiments, the provided methods alter or reduce the effects of T cell inhibitory pathways or signals in the tumor microenvironment. The methods counteract the upregulation and/or expression of inhibitory checkpoint receptors or ligands on source-specific exosomes, wherein the particular inhibitory checkpoint receptors or ligands on exosomes from particular cell types can negatively control T cell activation, T cell function, and T cell anti-cancer responses. For example, expression of certain immune checkpoint proteins (e.g., PD-1 or PD-L1) on exosomes from macrophages can reduce the potency and efficacy of immune cells by themselves or in conjunction with another anti-cancer therapy, such as a checkpoint inhibitor therapy. Such inhibitory pathways may otherwise impair certain desirable effector functions. Such inhibitory pathways may bypass the administered checkpoint inhibitor therapy.

The present invention is based on the discovery that somatic cells secrete a high level of exosomes that carry checkpoint proteins on their surface. The present invention is further based on the discovery that the levels of exosome-delivered checkpoint proteins from particular somatic cell types, such as macrophages, (i.e. macrophage-derived exosomes) can predict whether a patient will respond to anti-cancer medications, including checkpoint inhibitor therapies and further including antibodies that prevent the signaling of checkpoint inhibitor proteins. Further still, the present invention is based on the discovery that the levels of exosome-delivered proteins from particular cell types can predict whether a patient will respond to particular anticancer medications, including particular checkpoint inhibitor therapies and further including antibodies that prevent the signaling of particular checkpoint proteins.

Since the levels of exosome-delivered checkpoint proteins from particular cell types can predict whether a patient will or will not respond to a particular anticancer medication, the present invention provides for the administration of alternative anticancer medications. The present invention also provides for binding of at least one of the immunosuppressive exosomes or exosome-delivered immunosuppressive checkpoint proteins derived from the particular somatic and cancer cells within a subject's tissue or fluids, including blood, to treat cancer or to increase the efficacy of an anti-cancer medication. The present invention also provides for the replacement of at least one of the immunosuppressive exosomes or exosome-delivered immunosuppressive checkpoint proteins with immunostimulatory molecules, including immunostimulatory proteins, which will be bound to exosomes, including exosomes that have predictive source-specific markers. These immunostimulatory molecules bound to exosomes also include switch receptors that comprise the extracellular (or extraexosomal) binding domain of an inhibitory signaling receptor molecule (i.e. PD-1, TIGIT, CTLA-4, TIM3, CD160, BTLA) and the extracellular (or luminal with respect to the exosome) signaling domain of stimulatory molecule (i.e. an extracellular binding domain of CD40, OX40L, CD70, B7-H2, TL1A GITRL, CD58, CD48).

### A. Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics, and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably +5%, even more preferably 10%, and still more preferably +0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one of ordinary skill in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated, synthesized, or can be derived from a biological sample, which is defined infra.

As used herein, the term “autologous” is meant to refer to any material derived from the same individual to which it is later to be re-introduced into the individual.

A “biomarker” or “sensitivity marker” is a marker that is associated with differential sensitivity or response to a treatment, for example a treatment that includes anti-PD-1 and/or anti-PD-L1 therapy. Biomarkers can include checkpoint proteins, such as PD-1, and source-specific markers, such as CD11b. In some instances, the combination of particular checkpoint proteins and source-specific markers that are on the same exosome is of particular importance. Without wishing to be bound by a particular theory, it is believed that checkpoint proteins released on or within exosomes by particular cell types, such as macrophages, are more effective at immunomodulation and concomitantly a patient's sensitivity to a particular cancer treatment than the same checkpoint proteins released by another cell type, such as a cancer cell or an epithelial cell. Thus, the identification of the combination of particular checkpoint proteins and source-specific markers are believed to be helpful for predicting a patient's success to a particular cancer treatment, including a particular checkpoint inhibitor therapy.

Such biomarkers may include, but are not limited to, nucleic acids, proteins encoded thereby, or other small molecules. These markers can be used to advantage to identify those patients likely to respond to therapy from those that are unlikely to respond. They can also be targeted to modulate the response to therapy or used in screening assays to identify agents that have efficacy or act synergistically for the treatment and management of melanoma or other cancers. Detection of biomarkers can be carried out by standard histological and/or immuno-detection methods. In particular embodiments, the markers can be detected by any means of polypeptide detection, or detection of the expression level of the polypeptides. For example, the polypeptide can be detected using any of antibody detection methods (e.g., immunofluorescent (IF) methods, flow cytometry, and fluorescence activated cell sorting (FACS)), antigen retrieval and/or microarray detection methods can be used. A reagent that specifically binds to a marker polypeptide, e.g., an antibody, an antibody derivative, and an antibody fragment, can be used. Other detection techniques that can be used include, e.g., capture assays (e.g., ELISA), mass spectrometry (e.g., LCMS/MS), and/or polymerase chain reaction (e.g., RT-PCR).

Biomarkers can also be detected by systemic administration of a labeled form of an antibody to the biomarker, followed by imaging. In yet another approach, the method of detection of exosomal checkpoint proteins, e.g. PD-L1 protein, includes a fluorescent or quantum dot labeled antibody, such as labeled anti-checkpoint protein, e.g. anti-PD-L1, for labeling proteins on the surface of exosomes. After labeling the exosomal proteins, light scattering-based nanoparticle tracking analysis (NTA) employing a NANOSIGHT® NS300 Analyzer can be utilized to monitor individual vesicles as small as 10 nm. This combination of protein labeling and size measurement provides the unique ability to visualize exosomal proteins in suspension and directly observe their Brownian motion, yielding rapid, accurate, high-resolution sizing data by number distribution, as well as count and concentration measurements with visual confirmation of data analysis.

In another embodiment, a nucleic acid biomarker sample from the subject is evaluated by a nucleic acid detection technique as described herein. In other embodiments, biomarkers are measured or detected by measuring mRNA expression. Numerous techniques such as qRT-PCR, Fluidigm, RNAseq (e.g. ILLUMINA®), AFFYMETRIX® gene profiling, the NANOSTRING® NCOUNTER® platform, or NANOPORE® sequencing (OXFORD NANOPORE TECHNOLOGIES®) may be used by the person skilled in the art using their common general knowledge to measure RNA levels.

A “cancer protein” or “cancer marker protein” is a protein which is associated with malignant transformation and progression. Such proteins may or may not contain mutations. Exemplary “cancer proteins” include, without limitation, BRAF, NRAS, KIT, TP53, PTEN, EGFR, HER2, ALK, AKT1, KRAS, MET, RET, RHOA, ARID 1 A, CDH1, Akt, Wnt5A, MAPK1, HSP70, TRAP1, HSP90, SerpinH1, VEGFC, R-RAS, and HLA-G5.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil, and the like. Water or aqueous saline solutions and aqueous dextrose and glycerol solutions are preferably employed as carriers, particularly for injectable solutions. The compositions can be incorporated into particulate preparations of polymeric compounds such as polylactic acid, polyglycolic acid, etc., or into liposomes or micelles. Such compositions may influence the physical state, stability, rate of in vivo release, and rate of in vivo clearance of components of a pharmaceutical composition of the present invention. The pharmaceutical composition of the present invention can be prepared, for example, in liquid form, or can be in dried powder form (e.g., lyophilized). Suitable pharmaceutical carriers are described in “Remington's Pharmaceutical Sciences” by E.W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

A “checkpoint protein” or “immunomodulator protein” is a protein which plays a role in immune processes which regulate immune responses and in particular adaptive immune responses including those to malignant transformation and progression. A checkpoint protein may be immunosuppressive or immunostimulatory and the immunosuppression and immunostimulation of a particular checkpoint protein may further depend upon the context in which the checkpoint protein is presented to the adaptive immune cell, including whether a particular co-signaling protein, such as a source-specific protein, is co-presented. Such checkpoint proteins, include without limitation CD109, CD151, CD276, CD44, CD46, CD47, CD55, CD58, CD59, CD70, CD9, CD95, CD97, CD99, and B7H4.

A “chemotherapeutic agent,” “anti-cancer agent,” or “anti-cancer medication” is a chemical compound useful in the treatment of cancer. Classes of chemotherapeutic agents include, but are not limited to: alkylating agents, antimetabolites, kinase inhibitors, spindle poison plant alkaloids, cytotoxic/antitumor antibiotics, topoisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, RAF inhibitors, anti-sense oligonucleotides that that inhibit expression of genes implicated in abnormal cell proliferation or tumor growth. Chemotherapeutic agents useful in the treatment methods of the present invention can include agents that selectively inhibits one or more vital steps in signaling pathways, in the normal function of cancer cells, thereby leading to apoptosis.

Signal transduction inhibitors (STIs) include, but are not limited to, (i) bcr/abl kinase inhibitors such as, for example, STI 571 (Gleevec); (ii) epidermal growth factor (EGF) receptor inhibitors such as, for example, kinase inhibitors (Iressa, SSI-774) and antibodies (Imclone: C225 [Goldstein et al. (1995), Clin Cancer Res. 1: 1311-1318], and Abgenix: ABX-EGF); (iii) her-2/neu receptor inhibitors such as, for example, Herceptin™ (trastuzumab), and farnesyl transferase inhibitors (FTI) such as, for example, L-744,832 (Kohl et al. (1995), Nat Med. 1(8):792-797); (iv) inhibitors of Akt family kinases or the Akt pathway, such as, for example, rapamycin (see, for example, Sekulic et al. (2000) Cancer Res. 60:3504-3513); (v) cell cycle kinase inhibitors such as, for example, flavopiridol and UCN-01 (see, for example, Sausville (2003) Curr. Med. Chem. Anti-Canc Agents 3:47-56); and (vi) phosphatidyl inositol kinase inhibitors such as, for example, LY294002 (see, for example, Vlahos et al. (1994) J. Biol. Chem. 269:5241-5248). In a particular embodiment, the STI is selected from the group consisting of STI 571, SSI-774, C225, ABX-EGF, trastuzumab, L-744,832, rapamycin, LY294002, flavopiridal, and UNC-01. In yet another embodiment, the STI is L-744,832 chemotherapeutic agents which function as alkylating agents include without limitation, nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide).

Anti-cancer agents include checkpoint inhibitor therapies include without limitation PD-1/PD-L1 directed therapies, which further include without limitation pembrolizumab, nivolumab, atezolizumab, avelumab, durvalumab and other anti-PD-1/PD-L1 antibodies that are in development.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus, if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule. Complementary nucleic acid sequences may form the basis for molecular approaches to inhibition therapies, including checkpoint inhibitor therapies. For example, antisense oligonucleotides are generally complementary to the mRNA sequences and by binding to the mRNA sequence they prevent the translation of the information in the mRNA sequence into the amino acid sequence of a protein.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For instance, one common formula for calculating the stringency conditions required to achieve hybridization between nucleic acid molecules of a specified sequence homology is set forth below (Sambrook et al., Molecular Cloning, Cold Spring Harbor Laboratory (1989)):

Tm=81.5° C.+16.6 Log [Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tm is 57° C. The Tm of a DNA duplex decreases by 1-1.5° C. with every 1% decrease in homology. Thus, targets with greater than about 75% sequence identity would be observed using a hybridization temperature of 42° C. The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tm of the hybrid. Wash conditions should be as stringent as possible for the degree of identity of the probe for the target. In general, wash conditions are selected to be approximately 12-20° C. below the Tm of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in IX SSC and 0.5% SDS at 65° C. for 15 minutes. A very high stringency hybridization is defined as hybridization in 6×SSC, 5×Denhardt's solution, 0.5% SDS and 100 μg/mL denatured salmon sperm DNA at 42° C., and washed in 0.1×SSC and 0.5% SDS at 65° C. for 15 minutes. The ability of two nucleic acid sequences to hybridize may form the basis for molecular approaches to inhibition therapies, including checkpoint inhibitor therapies. For example, antisense oligonucleotides need not be exactly complementary to a target mRNA sequences, but instead may be able to still hybridize with the target mRNA sequence. By hybridizing to the mRNA sequence, antisense oligonucleotides prevent the translation of the information in the mRNA sequence into the amino acid sequence of a protein.

The phrase “consisting essentially of when referring to a particular nucleotide or amino acid means a sequence having the properties of a given SEQ ID NO:. For example, when used in reference to an amino acid sequence, the phrase includes the sequence per se and molecular modifications that would not affect the functional and novel characteristics of the sequence.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. Preferably, the epitope is about 4-18 amino acids, more preferably about 5-16 amino acids, and even more most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present invention can be an epitope.

As used herein, the term “exogenous” refers to any material introduced from or produced outside an organism, cell, tissue, or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide. In some embodiments, molecular approaches to checkpoint inhibitor therapy may involve administration of the molecular approach within an expression vector, i.e. administering an siRNA within a recombinant polynucleotide encapsulated within an adeno-associated virus.

The terms “exosome” and “extracellular vesicle”, are used interchangeably herein to describe membrane vesicles of endosomal and plasma membrane origin which are released from many different cell types. These extracellular vesicles (EVs) represent an important mode of intercellular communication by serving as vehicles for transfer between cells of membrane and cytosolic proteins, lipids, and RNA. Extracellular vesicles or exosomes are released from cells upon fusion of an intermediate endocytic compartment, the multivesicular body (MVB), with the plasma membrane. This liberates intraluminal vesicles (ILVs) into the extracellular milieu and the vesicles thereby releasing exosomes. There are other types of microvesicle, including apoptotic bodies and ectosomes, which are derived from cells undergoing apoptosis and plasma membrane shedding, respectively. Although apoptotic bodies, ectosomes, and exosomes are all roughly the same size (typically 40-100 nm) and all also contain ‘gulps’ of cytosol, they are different species of vesicles.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immune response” as used herein is defined as a cellular response to an antigen that occurs when lymphocytes identify antigenic molecules as foreign and induce the formation of antibodies and/or activate lymphocytes to remove the antigen.

The term “immunosuppressive” is used herein to refer to reducing overall immune response.

“Insertion/deletion”, commonly abbreviated “indel,” is a type of genetic polymorphism in which a specific nucleotide sequence is present (insertion) or absent (deletion) in a genome.

“Isolated” as used herein means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Regarding nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form. By the use of the term “enriched” in reference to nucleic acid it is meant that the specific DNA or RNA sequence constitutes a significantly higher fraction (2-5 fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁶-fold purification of the native message. Thus, purification of at least one order of magnitude, preferably two or three orders, and more preferably four or five orders of magnitude is expressly contemplated. Thus, the term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

The term “knockdown” as used herein refers to a decrease in gene expression of one or more genes.

The term “knockout” as used herein refers to the ablation of gene expression of one or more genes.

A “label” or a “detectable moiety” in reference to a nucleic acid or protein, example, refers to a composition that, when linked with a nucleic acid or protein, renders the nucleic acid or protein detectable, for example, by spectroscopic, photochemical, biochemical, immunochemical, or chemical means. Exemplary labels include, but are not limited to, radioactive isotopes, magnetic beads, metallic beads, colloidal particles, fluorescent dyes, enzymes, biotin, digoxigenin, haptens, and the like. A “labeled nucleic acid or oligonucleotide probe” is generally one that is bound, either covalently, through a linker or a chemical bond, or noncovalently, through ionic bonds, van der Waals forces, electrostatic attractions, hydrophobic interactions, or hydrogen bonds, to a label such that the presence of the nucleic acid or probe can be detected by detecting the presence of the label bound to the nucleic acid or probe.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo. An anti-cancer medication may be a molecular approach delivered by a lentivirus that causes the downregulation of a protein or may deliver to a cell genetic information causing the cell to express a protein which inhibits a checkpoint protein (e.g. a dominant negative construct) or causes a particular cell type to release exosomes encoding a immunostimulatory protein or switch receptor.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides. It will be understood that when a nucleotide sequence is represented by a DNA sequence (i.e., A, T, C, G), this also includes an RNA sequence (i.e., A, U, C, G) in which “U” replaces “T.”

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“PD-L1” may refer to human PD-L1 or homologs in other organisms, depending on the context in which it is used. Human PD-L1 is also known as CD274, B7-H, B7H1, B7-H1, B7 homolog 1, MGC142294, MGC142296, PDCD1L1, PDCD1LG1, PDCD1 ligand 1, PD-L1, Programmed cell death 1 ligand 1 and Programmed death ligand 1 and has Uniprot number Q9NZQ7 and NCBI gene ID number 29126. Human PD-L1 is a 290 amino acid type I transmembrane protein encoded by the CD274 gene on human chromosome 9. Mouse PD-L1 has NCBI GenBank ID number ADK70950.1.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “primer” as used herein refers to an oligonucleotide, either RNA or DNA, either single stranded or double stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15 to 25, 30, 50, 75 or more nucleotides nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complimentary bases may be interspersed within the oligonucleotide primer sequence, provided that the primer sequence has sufficient complementarity with the sequence of the desired template strand to functionally provide a template primer complex for the synthesis of the extension product.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single stranded or double stranded. The exact length of the probe will depend upon many factors, including temperature, source of probe and use of the method. For example, for diagnostic applications, depending on the complexity of the target sequence, the oligonucleotide probe typically contains 15 to 25, 30, 50, 75, or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically. The probe many also be labeled with a non-naturally occurring label to ease detection of the target.

“Sample,” “patient sample,” or “biological sample” generally refers to a sample which may be tested for a particular molecule, preferably a sensitivity marker molecule. A biological sample may include, but is not limited to a tissue sample, a tumor sample, a cell, or a biological fluid. A biological fluid may include, without limitation, blood, lymphatic fluid, aspirates, urine, saliva, tears, pleural fluids interstitial fluids, renal filtrates, nervous system fluids including lumbar puncture fluids, and the like, and may include combinations thereof.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran, or agarose.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

A “specific binding pair” comprises a specific binding member (sbm) and a binding partner (bp) which have a particular specificity for each other (or “specifically bind”) and which in normal conditions bind to each other in preference to other molecules. Examples of specific binding pairs are antigens and antibodies, ligands and receptors, and complementary nucleotide sequences. The ordinarily skilled person is aware of many other examples. Further, the term “specific binding pair” or “specific binding” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long. In general, the detection of immune complex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149; and 4,366,241, each incorporated herein by reference. Of course, one may find additional advantages through the use of a secondary binding ligand such as a second antibody and/or a biotin/avidin ligand binding arrangement, as is known in the art.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a nucleic acid sequence that defines a portion of a nucleic acid to which a binding molecule may specifically bind under conditions sufficient for binding to occur. In some embodiments, a target sequence refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (a) and beta (p) chain, although in some cells the TCR consists of gamma and delta (y/S) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

“Transplant” refers to a biocompatible lattice or a donor tissue, organ, or cell to be transplanted. An example of a transplant may include but is not limited to skin cells or tissue, bone marrow, and solid organs such as heart, pancreas, kidney, lung, and liver. A transplant can also refer to any material that is to be administered to a host. For example, a transplant can refer to a nucleic acid, a protein, or an exosome, including the proteins and nucleic acids bound thereon or therein.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “failing to respond to” treatment or “failing to respond to an anti-cancer medication” or “a failure to respond to” said treatment or said anti-cancer medication includes no reductions in the frequency or severity of at least one sign or symptom of a disease or disorder by the subject. Said “failing” or “failure” also includes deleterious responses including an acceleration of tumor growth or metastasis. “Failing” or “failures” may be determined by observing the patient longitudinally, by treating the subject with a placebo to compare the efficacy of the anti-cancer medication to that of the placebo, wherein even if the subject shows improvement in the reduction or severity of at least one sign of symptom of the disease or disorder with the treatment of the anti-cancer medication, if the same improvements are demonstrated with the placebo and if the improvements between the placebo and anti-cancer medication are not statistically significant based on single-subject study designs, the subject has failed to respond. “Failing” or a “failure” to respond may also be determined by comparing the subject to a population of subjects. If the subject's reductions in the frequency or severity of at least one sign or symptom of a disease or disorder, fall within the lower 30%, 20%, 10%, 5%, 4%, 3%, 2%, or 1% of the population based on the population's reductions in the frequency or severity of the same sign or symptom of the disease or disorder, the subject may considered as failing to respond to treatment.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. Methods of Treating for Checkpoint Inhibitor Compensation Caused by Bypassing Checkpoint Proteins on Exosomes from Particular Somatic and Cancerous Cell Types and Exosomes with Particular Source-Specific Markers

One of the major problems limiting the effects of anti-cancer therapies is that the subject's immune system must still initiate a response to the cancer being treated with the anti-cancer therapy. T cells are exhausted after persistent stimulation by checkpoint proteins from cancer cells and, as disclosed herein, the subject's somatic cells. The present invention is based on the discovery that the subject's somatic cells release exosomes containing said checkpoint proteins and that exosomes derived from certain cell types or exosomes containing certain source-specific markers are more influential than exosomes from other cells. For example, checkpoint proteins on exosomes derived from certain cell types and checkpoint proteins on exosomes further comprising source-specific markers are more influential than the same checkpoint protein on an exosome from a different somatic cell or the same checkpoint protein on an exosome comprising a different source-specific marker. Exhausted T cells have reduced effector functions such as production of cytokines and cytotoxicity against tumor cells, and they, in turn, express higher levels of checkpoint inhibitory molecules, such as PD-1 and CTLA-4, leading to a feed-forward mechanism caused by the exposure to the initial checkpoint protein on the exosome. PD-1 and CTLA-4 antibodies have been used clinically to treat multiple types of cancers. However, the majority of patients do not benefit significantly from these therapies. It has been shown that the genome-wide epigenetic landscape of exhausted T cells is different from that of effector T cells and memory T cells, and these exhausted T cells cannot be remodeled/reinvigorated by, e.g., PD-L1 blockade.

Accordingly, it is important to assess the levels of checkpoint proteins on particular exosomes before and/or during treatment to identify alternative compensatory checkpoint protein pathways that bypass the treatment. That way, the T cell exhaustion that was initiated by said compensatory checkpoint protein pathways can be prevented before the epigenetic landscape of the exhausted T cell is changed and/or the T cells cannot be remodeled/reinvigorated from their exhausted state. In some embodiments, the methods may provide for the T cells to be reinvigorated by checkpoint inhibitor therapies, i.e. antibodies (e.g., anti-PD-1, anti-CTLA-4, anti-PD-L1 antibodies).

In one aspect, a method for treating a patient failing to respond to a first checkpoint inhibitor therapy is provided. In one embodiment, the method comprises administering a second checkpoint inhibitor therapy to the patient after assessing a first biological sample and a second biological sample. In one embodiment, the first biological sample is obtained from the patient before the administration of the first checkpoint inhibitor therapy. In one embodiment, the second biological sample is obtained from the patient after the patient has been administered at least one treatment of the first checkpoint inhibitor therapy. In one embodiment, the first biological sample is obtained from the patient after at least one treatment of the first checkpoint inhibitor therapy, the second biological sample is obtained from the patient after the patient has been administered a subsequent treatment of the first checkpoint inhibitor therapy. In one embodiment, the first checkpoint inhibitor therapy comprises an inhibitor of signaling of a first checkpoint protein. In one embodiment, each of the first and second biological samples comprises an extracellular vesicle that has a source-specific marker, wherein the extracellular vesicle that has a source-specific marker comprises a second checkpoint protein. In one embodiment, in the assessing, the amount of the second checkpoint protein on the extracellular vesicle that has the source-specific marker of the second biological sample is elevated above or decreased below the amount of the second checkpoint protein on the extracellular vesicle that has the source-specific marker of the first biological sample. In one embodiment, the second checkpoint inhibitor therapy comprises an inhibitor of signaling of the second checkpoint protein. In such an embodiment, the second checkpoint protein is optionally an immunosuppressive protein. In one embodiment, the method comprises administering a second checkpoint stimulatory therapy to the patient after assessing a first biological sample and a second biological sample. In one embodiment, the second checkpoint stimulatory therapy comprises administering extracellular vesicles comprising the second checkpoint protein and the source-specific marker. In such an embodiment, the second checkpoint protein is optionally an immunostimulatory protein.

Source-Specific Markers

An ordinarily skilled artisan would be able to identify source-specific markers (or “markers” for short) as being proteins and carbohydrates that are produced by a particular cell type and are thereby indicative of said cell type from which the exosome was derived. The proteins may include those with post-translational modifications such as glycosylations, lipofications, etc. The source-specific marker may be a broad marker, i.e. a marker common to all cells of epidermal origin, endodermal origin, or mesodermal origin. The source-specific marker may be a specific source-specific marker, i.e. a marker that only identifies antigen presenting cells, macrophages, microglia, T cells, helper T cells, cytotoxic T cells, etc. The source-specific marker may be an intermediate marker, identifying cells derived from a specific non-pluripotent stem cells, i.e. bone marrow cells, myeloid cells, multi-potential hematopoietic stem cells, common myeloid progenitor cells, common lymphoid progenitor cells, embryonic yolk cells, peripheral blood monocyte cells, etc.

A non-limiting list of source-specific markers includes a marker for bone marrow derived antigen presenting cells, a marker for stroma cells, a marker for T cells, a marker for B cells, a marker for cancer cells, a marker for liver cells, a marker for stomach cells, a marker for pancreatic cells, a marker for small intestine cells, a marker for large intestine cells, a marker for endocrine cells, a marker for epithelial cells, a marker for endothelial cells, a marker for mesodermal cells, a marker for humoral cells, a marker for bone marrow, a marker for bone cells, a marker for nervous system cells, a marker for brain cells, a marker for neurons, a marker for glial cells, a marker for astrocytes, a marker for microglia, a marker for ependymal cells, a marker for stem cells, a marker for smooth muscle cells, a marker for cardiac cells, a marker for cardiac muscle cells, and a marker for basal cells.

In one embodiment, the marker for bone derived antigen presenting cells comprises a marker for macrophages. In one embodiment, the marker for macrophages comprises a marker for M2 macrophages, M1 macrophages, or tumor-associated macrophages. In one embodiment, the marker for macrophages comprises CD163, CD115, or CD11b. In one embodiment, the source-specific marker comprises CD163 and CD115. In one embodiment, the bone marrow derived antigen presenting cells comprises M2 macrophages or tumor-associated macrophages. In one embodiment, the marker for T cells comprises a marker for CD8+ T cells, a marker for CD4+ T cells, or a marker for regulatory T cells. In another embodiment, the marker for stromal cells comprises CD44, CD90, CD105, or Fibroblast Surface Antigen (SFA).

Since a single source-specific marker might not be able to identify the entirety of cells within a particular genus of cells (i.e. pancreatic cells), an ordinarily skilled artisan would understand that “a source-specific marker” within the present method may include two or more source-specific markers, the two or more source-specific markers being able to better capture/represent the entirety of a population of a genus of cells when used in combination to broaden the exosomes being measured (i.e. one source-specific marker identifying 50% of macrophages and another source-specific marker identifying the remaining 50% of the macrophages or one source-specific marker being used to capture 50% of exosomes from macrophages and another source-specific marker being used to capture the remaining 50% of exosomes from macrophages).

Reciprocally, since a single source-specific marker might not only identify cells within a particular genus but also the cells within another genus, an ordinarily skilled artisan would understand that “a source-specific marker” within the present method may include the intersection of two or more source-specific markers, the cell type being assessed having all of the two or more source-specific markers. An ordinarily skilled artisan would understand that all of the two or more source-specific markers are able to better capture/represent, when expressed together on an exosome, the cellular source of the exosome (i.e. wherein source-specific marker X identifies macrophages and natural killer cells, wherein source-specific marker Y identifies macrophages and basophils, and exosomes having both source-specific marker X and source-specific marker Y, thus, being exosomes derived from macrophages).

A non-limiting list of source-specific markers includes: CD63, CD9, C81, CD163, CD11b, CD11c, CD115, Gr-1, CD8, CD4, CD44, CD90, CD105, SFA, epidermal growth factor receptor (EGFR), vascular endothelial growth factor receptor (VEGFR), PD-L1, CD155, CD112, B7-1, B7-2, B7-H3, Galectin-9, Siglec-15, OX40 receptor ligand (OX40L), major histocompatibility complex class II molecules (MHC-II), ICAM-1, LFA-1, B7-H2, CD40, CD70, CD48, CD52, TL1A, GITRL, CD30L, T cell immunoglobin and mucin domain 1 (TIM1), TIM3, TIM4, signaling lymphocytic activation molecule (SLAM), tumor necrosis factor superfamily member 14 (TNFSF14 or LIGHT), herpesvirus entry mediator (HVEM), PD-1, cytotoxic T lymphocyte-associated protein (CTLA-4), T cell immunoreceptor with Ig and immunoreceptor tyrosine-based inhibitory motif domains (TIGIT), lymphocyte-activation gene 3 (LAG3), OX40, V-set and immunoglobulin domain containing 3 (VISG3), VISG8, inducible T cell costimulator (ICOS), CD28, CD40L, death receptor 3 (DR3), glucocorticoid-induced tumor necrosis factor-related protein (GITR), CD30, CD2, CD226, CD160, B and T lymphocyte attenuator (BTLA), programed death-1 homolog (PD-1H), leukocyte-associated immunoglobulin-like receptor 1 (LAIR1), and natural killer cell receptor 2B4 (NKCR2B4). An ordinarily skilled artisan would be able to identify other source-specific markers.

In some embodiments, the source-specific marker is one or more markers, which individually or in combination, quantify all exosomes in the biological sample; the source being all cells that secrete exosomes. For example, in combination CD9, CD63, and CD81 may be used as the markers for total exosomes in the biological sample by binding or identifying extracellular vesicles comprising CD9, extracellular vesicles comprising CD63, and extracellular vesicles comprising CD81. The combination of extracellular vesicles comprising CD9, extracellular vesicles comprising CD63, and extracellular vesicles comprising CD81 being the total amount of extracellular vesicles in a biological sample. In some embodiments, the source-specific marker excludes one or more markers, individually or which in combination, quantify all exosomes in the biological sample.

In some embodiments, the extracellular vesicle that has a source-specific marker is not from a cancer cell. In some embodiments, the source-specific marker excludes a marker of a cancer cell. In some embodiments, the source-specific marker excludes at least one of: a marker for bone marrow derived antigen presenting cells, a marker for stroma cells, a marker for T cells, a marker for B cells, a marker for liver cells, a marker for stomach cells, a marker for pancreatic cells, a marker for small intestine cells, a marker for large intestine cells, a marker for endocrine cells, a marker for epithelial cells, a marker for endothelial cells, a marker for mesodermal cells, a marker for humoral cells, a marker for bone marrow, a marker for bone cells, a marker for nervous system cells, a marker for brain cells, a marker for neurons, a marker for glial cells, a marker for astrocytes, a marker for microglia, a marker for ependymal cells, a marker for stem cells, a marker for smooth muscle cells, a marker for cardiac cells, a marker for cardiac muscle cells, or a marker for basal cells.

Checkpoint Proteins

In some embodiments, checkpoint proteins may be immunosuppressive proteins. In some embodiments, checkpoint proteins may be immunostimulatory. In some embodiments, whether a checkpoint protein is immunosuppressive or immunostimulatory may be context specific and depend upon the interaction of the particular immune cell type with the checkpoint protein or alternatively may depend upon whether particular co-signaling molecules are present. In some embodiments, co-signaling molecules may be other checkpoint proteins. In some embodiments, co-signaling molecules may be source-specific markers. Accordingly, in some embodiments, source-specific markers may be checkpoint proteins, and in some embodiments checkpoint proteins may be source-specific markers. In some embodiments, the source-specific marker and the checkpoint protein may be the same protein. In some embodiments, the source-specific marker and the checkpoint protein differ.

A non-exhaustive list of checkpoint proteins includes: PD-L1, CD155, CD112, B7-1, B7-2, B7-H3, Galectin-9, Siglec-15, OX40L, B7-H2, CD70, CD48, CD52, TL1A, GITRL, CD30L, TIM1, TIM3, TIM4, SLAM, LIGHT, HVEM, PD-1, CTLA-4, TIGIT, LAG3, OX40, VISG-3, VISG-8, ICOS, CD28, DR3, GITR, CD30, CD2, CD226, CD160, BTLA, PD1H, LAIR1, and NKCR2B4. An ordinarily skilled artisan would be able to identify checkpoint proteins, whether they are immunosuppressive or immunostimulatory, and if context-specific, the context in which they are immunosuppressive or immunostimulatory.

Without wishing to limit the following proteins to being solely immunosuppressive (i.e. without wishing to disclaim them as having context specific activity), a non-exhaustive list of immune regulatory proteins includes: PD-L1, CD155, CD112, B7-1, B7-H3, Galectin-9, Siglec-15, OX40L, B7-H2, CD48, CD52, TL1A, CD30L, TIM1, TIM3, TIM4, SLAM, LIGHT, HVEM, PD-1, CTLA-4, TIGIT, LAG3, VISG-3, VISG-8, CD28, CD40L, DR3, CD30, CD2, CD226, CD160, BTLA, PD1H, LAIR1, and NKCR2B4. Without wishing to limit the following checkpoint proteins to being solely immunostimulatory (i.e. without wishing to disclaim them as having context specific activity), the following is a non-exhaustive list of immunostimulatory (i.e. co-stimulatory) proteins: CD28, ICOS, CD40L, CD137/4-1BB, CD27, OX40, GITR, SIRPα, B7-1, ICOSLG, CD40, CD137L, CD70, OX40L, GITRL, and CD47.

Checkpoint Inhibitory Therapies

Checkpoint inhibitory therapies, including the first and second checkpoint inhibitory therapies, may include those that are commercially available and those that are presently not commercially available. Checkpoint inhibitory therapies may include anti-checkpoint protein antibodies, molecular approaches, and small molecule inhibitors. Molecular approaches may include, without limitation, siRNA, antisense RNA, RNAi, and CRISPR-Cas mediated downregulation, knockdown, or knockout approaches.

An ordinarily skilled artisan would be able to identify a checkpoint inhibitor therapy and would understand that the purpose of the checkpoint inhibitor therapy is to prevent the signaling of a ligand-receptor signaling system. Accordingly, a “checkpoint inhibitor therapy” comprises an inhibitor not only of the checkpoint protein of interest but also its reciprocal binding partner. For example, when the checkpoint protein is a receptor, the checkpoint inhibitor therapy may comprise an inhibitor of the receptor or its cognate ligand, thereby inhibiting the signaling of the receptor. When the checkpoint protein is a ligand, the checkpoint inhibitor therapy may comprise an inhibitor of the ligand or its cognate receptor, thereby inhibiting the signaling of the ligand. For example, if a checkpoint protein of interest is CD155, which binds to TIGIT, an inhibitor of either CD155 or TIGIT would be considered a checkpoint inhibitor therapy or an inhibitor of the signaling of CD155. An ordinarily skilled artisan would further understand that by convention a protein might be considered a “ligand” even though it has a transmembrane domain and an intracellular signaling domain and it functions as a receptor, e.g. PD-L1, even though said protein might also have a binding partner, where upon binding, it causes its binding partner to initiate an intracellular signaling response of its own, e.g. how PD-L1 binding to PD-1 initiates PD-1 signaling within the cell expressing the PD-1 on its surface.

In one embodiment, the second checkpoint protein is CD155 and the inhibitor of the signaling of the second checkpoint protein is a TIGIT inhibitor. Accordingly, in one embodiment, the second checkpoint protein is a receptor and the inhibitor of the signaling of the second checkpoint protein is an inhibitor of a ligand to the receptor. In another embodiment, the second checkpoint protein is a ligand and the inhibitor of the signaling of the second checkpoint protein is an inhibitor of a receptor to the ligand. In another embodiment, the checkpoint protein is a receptor and a ligand, e.g. PD-L1 or PD-1.

In some embodiments, the checkpoint inhibitory therapies include antibodies, which include, but are not limited to, an anti-PD-1, anti-CTLA-4, or anti-PD-L1 antibody. Examples of anti-PD-1 antibodies include, but are not limited to, pembrolizumab (KEYTRUDA®, formerly lambrolizumab, also known as MK-3475), and nivolumab (BMS-936558, MDX-1106, ONO-4538, OPDIVA®) or an antigen-binding fragment thereof. In certain embodiments, the modified cell may be administered in combination with an anti-PD-L1 antibody or antigen-binding fragment thereof. Examples of anti-PD-L1 antibodies include, but are not limited to, BMS-936559, MPDL3280A (TECENTRIQ®, Atezolizumab), and MEDI4736 (Durvalumab, Imfinzi). In certain embodiments, the modified cell may be administered in combination with an anti-CTLA-4 antibody or antigen-binding fragment thereof. An example of an anti-CTLA-4 antibody includes, but is not limited to, Ipilimumab (trade name Yervoy).

In one embodiment, the first checkpoint inhibitor therapy comprises an inhibitor of programmed cell death protein 1 (PD-1) or programmed death-ligand protein 1 (PD-L1). In a further embodiment thereof, the inhibitor of PD-L1 comprises atezolizumab.

A checkpoint inhibitor therapy may also include disrupting the expression of, for example and without limitation, the Adenosine A2A receptor (A2AR), B7-H3 (CD276), B7-H4 (VTCN1), the B and T Lymphocyte Attenuator protein (BTLA/CD272), CD96, the Cytotoxic T-Lymphocyte Associated protein 4 (CTLA-4/CD152), Indoleamine 2,3-dioxygenase (IDO), the Killer-cell Immunoglobulin-like Receptor (KIR), the Lymphocyte Activation Gene-3 (LAG3), the T cell immunoreceptor with Ig and ITIM domains (TIGIT), T-cell Immunoglobulin domain and Mucin domain 3 (TIM-3), or the V-domain Ig suppressor of T cell activation (VISTA).

Cancers

The types of cancers to be treated with the checkpoint inhibitor therapy of the invention include, carcinoma, blastoma, and sarcoma, and certain leukemia or lymphoid malignancies, benign and malignant tumors, and malignancies e.g., sarcomas, carcinomas, and melanomas. Other exemplary cancers include but are not limited breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, and the like. The cancers may be non-solid tumors (such as hematological tumors) or solid tumors. Adult tumors/cancers and pediatric tumors/cancers are also included. In one embodiment, the cancer is a solid tumor or a hematological tumor. In one embodiment, the cancer is a carcinoma. In one embodiment, the cancer is a sarcoma. In one embodiment, the cancer is a leukemia. In one embodiment the cancer is a solid tumor.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

Carcinomas that can be amenable to therapy by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

Sarcomas that can be amenable to therapy by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

In certain exemplary embodiments, the methods of the invention are used to treat a myeloma, or a condition related to myeloma. Examples of myeloma or conditions related thereto include, without limitation, light chain myeloma, non-secretory myeloma, monoclonal gamopathy of undertermined significance (MGUS), plasmacytoma (e.g., solitary, multiple solitary, extramedullary plasmacytoma), amyloidosis, and multiple myeloma. In one embodiment, a method of the present disclosure is used to treat multiple myeloma. In one embodiment, a method of the present disclosure is used to treat refractory myeloma. In one embodiment, a method of the present disclosure is used to treat relapsed myeloma.

In certain exemplary embodiments, the methods of the invention are used to treat a melanoma, or a condition related to melanoma. Examples of melanoma or conditions related thereto include, without limitation, superficial spreading melanoma, nodular melanoma, lentigo maligna melanoma, acral lentiginous melanoma, amelanotic melanoma, or melanoma of the skin (e.g., cutaneous, eye, vulva, vagina, rectum melanoma). In one embodiment, a method of the present disclosure is used to treat cutaneous melanoma. In one embodiment, a method of the present disclosure is used to treat refractory melanoma. In one embodiment, a method of the present disclosure is used to treat relapsed melanoma.

In yet other exemplary embodiments, the methods of the invention are used to treat a sarcoma, or a condition related to sarcoma. Examples of sarcoma or conditions related thereto include, without limitation, angiosarcoma, chondrosarcoma, Ewing's sarcoma, fibrosarcoma, gastrointestinal stromal tumor, leiomyosarcoma, liposarcoma, malignant peripheral nerve sheath tumor, osteosarcoma, pleomorphic sarcoma, rhabdomyosarcoma, and synovial sarcoma. In one embodiment, a method of the present disclosure is used to treat synovial sarcoma. In one embodiment, a method of the present disclosure is used to treat liposarcoma such as myxoid/round cell liposarcoma, differentiated/dedifferentiated liposarcoma, and pleomorphic liposarcoma. In one embodiment, a method of the present disclosure is used to treat myxoid/round cell liposarcoma. In one embodiment, a method of the present disclosure is used to treat a refractory sarcoma. In one embodiment, a method of the present disclosure is used to treat a relapsed sarcoma.

Normalizing for Total Exosome Levels

An ordinarily skilled artisan would understand that biological samples can vary based on the total amount of protein, total amount of exosomes, total number of cells, number of particular cell types (i.e. red blood cells, white blood cells), the number of platelets, the amount of liquid, the amount of elements, etc. For example, a patient may experience loss of appetite as an anti-cancer medication regime progresses, and accordingly, blood protein levels might decrease after treatment. Alternatively, a patient may be administered a saline infusion before a biological sample is taken. Alternatively, the total amount of exosomes in the blood may vary across biological samples. An ordinarily skilled artisan would thus understand that the assessing of two or more biological samples may involve normalizing for one or more variables changing between the samples while determining the amount of checkpoint protein on an exosome having a source-specific marker of interest. For example, a subject could have a 50% increase in a second biological sample in the amount of CD155, as a checkpoint protein, on exosomes with CD163, as a source-specific marker, compared to the same measure in the first biological sample, but the second biological sample also has a 50% increase in the total number of exosomes compared to the first biological sample. Accordingly, the 50% increase in the second biological sample in the amount of CD155 on CD163 containing exosomes could be attributable to the 50% increase in the total number of exosomes, and the normalization would, thus, account for the 50% increase in the total number of exosomes.

Normalization may be accounted for by dividing the amount of the checkpoint protein on the exosomes having the source-specific protein by the variable being normalized (e.g. the total number of exosomes). Normalization may be accounted for by performing a correlation of all the samples between the amount of the checkpoint protein on exosomes having the source-specific protein of interest and the value being normalized (e.g. correlating said levels of checkpoint protein on source-specific marker-containing exosomes with the total number of exosomes in the biological samples). When the normalization is a correlation, the adjusted value of the levels of the checkpoint protein on source-specific marker-containing exosomes may be obtained from the correlation/regression analysis, and the adjusted value would be the normalized variable used in the assessing of the first and second biological samples. Methods of normalizing for the total amount of a protein, total amount of exosomes, total number of cells, number of particular cell types (i.e. red blood cells, white blood cells), the number of platelets, the amount of liquid, and the amount of elements are known in the art. For example, a Bradford assay may be used to identify the total amount of protein in a biological sample.

For illustrative purposes, the total amount of exosomes can be determined by labelling, binding, and/or sequestering: extracellular vesicles comprising CD9, extracellular vesicles comprising CD63, and/or extracellular vesicles comprising CD81.

Increases and Decreases in the Assessing

As noted above, the first and second biological samples are assessed for relative increases and decreases in the amount of the second checkpoint protein on the extracellular vesicle that has the source-specific marker. In some embodiments, the amount of the second checkpoint protein on the extracellular vesicle that has the source-specific marker of the second biological sample is elevated at least 10%, 20%, 30%, 40%, 50%, 60%, 70%, 80%. 90%, 1 fold, 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 33 fold, 100 fold, 333 fold, 1000 fold, 3333 fold, or 10,000 fold above the amount of the second checkpoint protein on the extracellular vesicle that has the source-specific marker of the first biological sample, and the second checkpoint inhibitor therapy comprising an inhibitor of signaling of the second checkpoint protein. In these embodiments, the second checkpoint protein is optionally an immunosuppressive protein and the second checkpoint inhibitor therapy optionally comprises an inhibitor of signaling of the second checkpoint protein.

In some embodiments, the amount of the second checkpoint protein on the extracellular vesicle that has the source-specific marker of the second biological sample is decreased at least 10%, 20%, 30%, 40%, 50%, 60%, 70%, 80%. 90%, 1 fold, 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 33 fold, 100 fold, 333 fold, 1000 fold, 3333 fold, or 10,000 fold below the amount of the second checkpoint protein on the extracellular vesicle that has the source-specific marker of the first biological sample, and the second checkpoint inhibitor therapy comprising an inhibitor of signaling of the second checkpoint protein. In these embodiments, the second checkpoint protein is optionally an immunostimulatory protein and the method optionally comprises administering a second checkpoint stimulatory therapy to the patient after assessing a first biological sample and a second biological sample. In such an embodiment, the second checkpoint stimulatory therapy optionally comprises administering extracellular vesicles comprising the second checkpoint protein and the source-specific marker.

Administration of Anti-Cancer Medications, Including Checkpoint Inhibitor Therapies

The anti-cancer medication of the present invention can be administered by any suitable route, for example, by injection, by oral, pulmonary, topical or other modes of administration. In general, pharmaceutical compositions of the present invention, comprise, among other things, pharmaceutically acceptable diluents, preservatives, solubilizers, emulsifiers, adjuvants and/or carriers. Such compositions can include diluents of various buffer content (e.g., Tris-HCl, acetate, phosphate), pH and ionic strength; and additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol). The compositions can be incorporated into particulate preparations of polymeric compounds such as polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington's Pharmaceutical Sciences, 18th Ed. (1990, Mack Publishing Co., Easton, Pa. 18042) pages 1435-1712 which are herein incorporated by reference. The pharmaceutical composition of the present invention can be prepared, for example, in liquid form, or can be in dried powder form (e.g., lyophilized).

In yet another embodiment, the anti-cancer medication of the present invention can be delivered in a controlled release system, such as using an intravenous infusion, an implantable osmotic pump, a transdermal patch, liposomes, or other modes of administration. In a particular embodiment, a pump may be used (see Langer, supra; Sefton, CRC Crit. Ref. Biomed. Eng. (1987) 14:201; Buchwald et al., Surgery (1980) 88:507; Saudek et al., N. Engl. J. Med. (1989) 321:574). In another embodiment, polymeric materials may be employed (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. (1983) 23:61; see also Levy et al., Science (1985) 228:190; During et al., Ann. Neurol. (1989) 25:351; Howard et al., J. Neurosurg. (1989) 71: 105). In yet another embodiment, a controlled release system can be placed in proximity of the target tissues of the animal, thus requiring only a fraction of the systemic dose (see, e.g., Goodson, in Medical Applications of Controlled Release, supra, (1984) vol. 2, pp. 115-138). In particular, a controlled release device can be introduced into an animal in proximity to the site of inappropriate immune activation or a tumor. Other controlled release systems are discussed in the review by Langer (Science (1990) 249:1527-1533).

Methods of Measuring the Checkpoint Protein and the Source-Specific Marker and Methods of Capturing Exosomes Comprising the Checkpoint Protein and the Source-Specific Marker

A non-limiting list of methods for measuring the checkpoint protein and source specific markers include immunoblotting (i.e. western blotting), semi-quantifiable and quantifiable immunohistochemistry, semi-quantifiable and quantifiable immunofluorescence, immunolabeling followed by transmission electron microscopy (TEM), NANOSIGHT® nanoparticle tracking systems, reverse phase protein arrays (RPPA), density gradient centrifugation optionally followed by an immunolabeling approach, enzyme-linked immunosorbent assay (“ELISA”), immunoglobin-conjugated bead labelling followed optionally by flow cytometry, ELISPOT, cytometric bead array or other multiplex methods, and other immunoaffinity-based methods. Immunolabelling followed by TEM may include immunogold-labeling with a monoclonal antibody (e.g. against the extracellular domain of PD-L1). An ordinary skilled artisan would be able to identify methods for detecting the levels of particular proteins.

Purification of exosomes may include the use of commercially available kits, the use of polymer based pull-down methods, the use of immunoglobin-conjugated bead pull-down methods, density gradient centrifugation and ultracentrifugation. Without limitation, density gradient centrifugation and ultracentrifugation may include the use of iodixanol, ficoll, sucrose, lithium, and other gradients and may include centrifugal forces between 16,500 G and 100,000 G.

### C. Methods of Modifying Treatments Based on Exosomal Predictive Markers

As discussed above, the present invention provides for methods, which in part measure the levels of checkpoint proteins on exosomes carrying a source specific marker and/or total exosome levels. As noted above, the present invention is based upon the discovery that particular checkpoint proteins released on exosomes derived from particular cells can provide signals that promote cancer or suppress anti-cancer medications.

Thus, in one embodiment, a method of identifying whether a subject is responsive to a first checkpoint protein inhibitor therapy is provided, the method comprising administering a checkpoint therapy that the patient was not predicted to be non-responsive to an anti-cancer medication, including without limitation, a checkpoint inhibitor therapy. In an embodiment, the method identifying whether a subject is responsive to a first checkpoint protein inhibitor therapy, comprises: i) detecting the levels of extracellular vesicle-bound second checkpoint protein obtained from the subject; ii) detecting the levels of a marker for the extracellular vesicle; and iii) identifying the subject as non-responsive to treatment wherein: a) levels of the extracellular vesicle-bound checkpoint protein and levels of the marker for the extracellular vesicle correlate in a population of individuals, and b) there is a X increase in the level of the extracellular vesicle-bound second checkpoint protein from the subject per the level of marker for the extracellular vesicle from the subject above Y, wherein Y equals the mean of Z and X is the standard deviation of Z, wherein Z is levels of the extracellular vesicle-bound second checkpoint protein of each individual from the population of individuals divided by levels of the marker for the extracellular vesicle of said each individual from the population of individuals, wherein X is at least 0.68, 1, 1.5, 2, 2.5, 3, 3.5, 4, 4.5, 5, 5.5, 6, 6.5, 7, 7.5, 8, 8.5, 9, 9.5, or 10.

In another embodiment, a method of identifying whether a subject is responsive to a first checkpoint protein inhibitor therapy is provided, the method comprising administering a checkpoint therapy that the patient was not predicted to be non-responsive to an anti-cancer medication, including without limitation, a checkpoint inhibitor therapy. In an embodiment, the method identifying whether a subject is responsive to a first checkpoint protein inhibitor therapy, comprises: i) detecting the level of a second checkpoint protein on extracellular vesicles comprising a first source-specific marker obtained from the subject; ii) detecting the level of a second marker or second source-specific marker for an extracellular vesicle; and iii) identifying the subject as non-responsive to treatment wherein: a) levels of the second checkpoint protein on extracellular vesicles comprising the first source-specific protein and the levels of the second marker or second source-specific marker for a extracellular vesicle correlate in a population of individuals, and b) there is a X increase in the level of the second checkpoint protein on extracellular vesicles comprising the first source-specific protein from the subject per the level of a second marker or second source-specific marker for an extracellular vesicle from the subject above Y, wherein Y equals the mean of Z and X is the standard deviation of Z, wherein Z is levels of the second checkpoint protein on extracellular vesicles comprising the first source-specific protein of each individual from the population of individuals divided by levels of a second marker or second source-specific marker for an extracellular vesicle of said each individual from the population of individuals, wherein X is at least 0.68, 1, 1.5, 2, 2.5, 3, 3.5, 4, 4.5, 5, 5.5, 6, 6.5, 7, 7.5, 8, 8.5, 9, 9.5, or 10.

Correlation and Regression Analysis

The correlation may be obtained from a population of subjects who are responders or may be obtained from the population of subjects who are responders and non-responders. The correlation may be a two-dimensional correlation between two variables (for example the correlation between the level of total exosomes and the level of PD-L1 on CD163 positive exosomes). In some embodiments, the correlation may be a multi-dimensional analysis involving more than two variables. By providing for more than two variables, the predictive ability of the correlation from the population may become more powerful. The predictive function becomes more powerful as the additional variables account for a greater amount of the variability in the system. For example, assume that the correlation between the levels of total exosomes and the levels of PD-L1 on CD163 positive exosomes results in an significant R² value of 0.4. The R² is also a measure of the percentage of variability accounted for by the levels of PD-L1 on CD163 positive exosomes in predicting total exosome levels. In the example, 40% of the variability in the levels of total exosomes is accounted for by the levels of PD-L1 on CD163 positive exosomes. The measure of 1- R² is the measure of all unaccounted for variability. In the example, 1- R² equals 0.6, and 60% of the variability in the levels of total exosomes is not accounted for by the levels of PD-L1 on CD163 positive exosomes. In this example, a multi-variable correlational analysis of the levels of CD155 on CD11b positive exosomes and the levels of PD-L1 on CD163 positive exosomes correlate, in combination, with the total exosome levels resulting in an R² of 0.75%. The addition of the second variable, the levels of CD155 on CD11b positive exosomes, thus increases the ability to predict the variability in total exosome levels by an absolute 35% (75%-40%). As illustrated, the correlation/regression analysis should have a significant correlational measure (e.g. a Pearson correlation coefficient). The significant correlational measure may vary depending upon the correlational/regression model being used.

Multivariate analysis may include linear modeling, automatic linear modeling, MANOVAs, ANCOVAS, structural equation modeling, and other approaches readily apparent to an ordinarily skilled artisan. Multivariate analysis may include an analysis of two or more biological samples from each subject in the population, including an analysis of biological samples before and after a single treatment of an anti-cancer medication, such as a checkpoint inhibitor. Multivariate analysis may include an analysis of two or more biological samples from each subject in the population, including an analysis of biological samples before and after intermediary treatments of an anti-cancer medication (e.g. before and after a second treatment). Several multivariate approaches can account for the inclusion of temporal information and two or more measurements from the same subject for the same variable and they include repeated measures ANCOVAS, repeated measures MANOVAs, and structural equation modeling, such as linear structural relations (LISREL). In some embodiments, the multivariate analysis may also include information not derived from exosomes, checkpoint proteins thereon, or source-specific markers thereon. Such information may include, but is not limited to, information from the subject or an individual of the population, such as weight, body mass index, tumor size, tumor load, disease stage, etc.

In some embodiments, the correlation/regression analysis may be linear, exponential, logarithmic, polynomial, etc. In some embodiments, each variable may be transformed logarithmically, exponentially, with a tangent, arc-tangent, cosine, arc-cosine, sine, arc-sine, a square root, by being brought to the 1/X power (e.g. the ½ power being the square root), etc.

In some embodiments, one or more standard deviations are determined, including for example and without limitation, the standard deviation of each measure and the standard deviation of the adjusted value of each data point, each data point adjusted so that its value is provided relative to the regression line or curve. In some embodiments, such as a linear regression, the adjusted value for each data point is the distance each data point is from the regression line, the distance being measured orthogonally to the slope of the line. In this embodiment, said standard deviation of the adjusted variable may be the standard deviation obtained from each and every orthogonal distance from line for each and every data point.

The size of the population of individuals used for finding the correlation can be determined by a person of ordinary skill in the art based on the assumptions and power analysis of the statistical test being used and based on the availability of clinical and preclinical data, samples, subjects, individuals of the population, patients, etc. Individuals from a population may also include post-mortem individuals and samples therefrom may include post-mortem samples, i.e. blood bank samples, brain bank samples, tissue bank samples.

Identifying an Increase or Decrease in a Subject being Tested Whether he is a Non-Responder

After finding a significant correlation from a population of patients, the subject must then be tested in the same manner as the population of patients. A corresponding biological sample may be obtained from the subject. By “corresponding” it is meant that the biological sample is the same type as that analyzed in the population for detecting the levels of the extracellular vesicle-bound checkpoint protein and marker for the extracellular vesicle (i.e. if a blood sample was taken from the population, a corresponding sample is a blood sample taken from the subject).

In one embodiment, the correlation/regression analysis may be a linear correlation/regression. In said embodiment, to obtain the adjusted value for this subject's level of the extracellular-bound checkpoint protein relative to the level of the marker for the extracellular vesicle (i.e. the orthogonal distance of the data point from the regressed line), the level of the extracellular-bound checkpoint protein may be divided by the level of the marker for the extracellular vesicle from the subject. In said embodiment, the adjusted value of the level of the extracellular-bound checkpoint protein relative to the level of the marker for the extracellular vesicle for each individual in the population may be obtained in the same manner, by dividing level of the extracellular-bound checkpoint protein by the level of the marker for the extracellular vesicle from each individual in the population. In said embodiment, the mean, median, or mode adjusted level of the extracellular-bound checkpoint protein to the level of the marker for the extracellular vesicle from each individual in the population may be obtained. In said embodiment, the standard deviation of the adjusted level of the extracellular-bound checkpoint protein to the level of the marker for the extracellular vesicle from each individual in the population may be obtained.

In some embodiments, the subject will be identified as a non-responder when the level of the extracellular-bound checkpoint protein divided by the level of the marker for the extracellular vesicle from the subject is at least 0.68, 1, 1.5, 2, 2.5, 3, 3.5, 4, 4.5, 5, 5.5, 6, 6.5, 7, 7.5, 8, 8.5, 9, 9.5, or 10 standard deviations of the population of individuals from the mean, median, or mode of the values obtained by dividing the level of the extracellular-bound checkpoint protein from the population of individuals by the level of the marker for the extracellular vesicle from the population of individuals.

In one embodiment, the correlation/regression analysis may be a linear correlation/regression. In said embodiment, to obtain the adjusted value for this subject's level of the second checkpoint protein on extracellular vesicles comprising the first source-specific protein relative to the level of a second marker or second source-specific marker for an extracellular vesicle (i.e. the orthogonal distance of the data point from the regressed line), the level of the second checkpoint protein on extracellular vesicles comprising the first source-specific protein may be divided by the level of a second marker or second source-specific marker for an extracellular vesicle from the subject. In said embodiment, the adjusted value of the level of the second checkpoint protein on extracellular vesicles comprising the first source-specific marker for each individual in the population relative to the level of a second marker or second source-specific marker for an extracellular vesicle for each individual in the population may be obtained in the same manner, by dividing the level of the second checkpoint protein on extracellular vesicles comprising the first source-specific marker by the level of a second marker or second source-specific marker for an extracellular vesicle from each individual in the population. In said embodiment, the mean, median, or mode of the adjusted level of the second checkpoint protein on extracellular vesicles comprising the first source-specific protein to the level of a second marker or second source-specific marker for an extracellular vesicle from each individual in the population may be obtained. In said embodiment, the standard deviation of the adjusted level of the second checkpoint protein on extracellular vesicles comprising the first source-specific protein to the level of a second marker or second source-specific marker for an extracellular vesicle from each individual in the population may be obtained.

In some embodiments, the subject will be identified as a non-responder when the level of the second checkpoint protein on extracellular vesicles comprising the first source-specific protein of the subject divided by the level of a second marker or second source-specific marker for an extracellular vesicle from the subject is at least 0.68, 1, 1.5, 2, 2.5, 3, 3.5, 4, 4.5, 5, 5.5, 6, 6.5, 7, 7.5, 8, 8.5, 9, 9.5, or 10 standard deviations of the population from the mean, median, or mode of the values obtained by dividing the levels of the second checkpoint protein on extracellular vesicles comprising the first source-specific protein of each individual from the population of individuals by the levels of a second marker or second source-specific marker for an extracellular vesicle.

In some embodiments, the marker for the extracellular vesicle comprises: CD63, CD9, C81, CD163, CD11b, CD11c, CD115, Gr-1, CD8, CD4, CD44, CD90, CD105, SFA, epidermal growth factor receptor (EGFR), vascular endothelial growth factor receptor (VEGFR), PD-L1, CD155, CD112, B7-1, B7-2, B7-H, Galectin-9, Siglec-15, OX40 receptor ligand (OX40L), major histocompatibility complex class II molecules (MHC-II), ICAM-1, LFA-1, B7-H2, CD40, CD70, CD48, CD52, TL1A, GITRL, CD30L, T cell immunoglobin and mucin domain 1 (TIM1), TIM3, TIM4, signaling lymphocytic activation molecule (SLAM), tumor necrosis factor superfamily member 14 (TNFSF14 or LIGHT), herpesvirus entry mediator (HVEM), PD-1, cytotoxic T lymphocyte-associated protein (CTLA-4), T cell immunoreceptor with Ig and immunoreceptor tyrosine-based inhibitory motif domains (TIGIT), lymphocyte-activation gene 3 (LAG3), OX40, V-set and immunoglobulin domain containing 3 (VISG3), VISG8, inducible T cell costimulator (ICOS), CD28, CD40L, death receptor 3 (DR3), glucocorticoid-induced tumor necrosis factor-related protein (GITR), CD30, CD2, CD226, CD160, B and T lymphocyte attenuator (BTLA), programed death-1 homolog (PD-1H), leukocyte-associated immunoglobulin-like receptor 1 (LAIR1), or natural killer cell receptor 2B4 (NKCR2B4). The marker for the extracellular vesicle herein may include the same markers as those source-specific markers in the method provided supra.

In some embodiments, the extracellular vesicle-bound immune regulatory proteins comprises: PD-L1, CD155, CD112, B7-1, B7-2, B7-H3, Galectin-9, Siglec-15, OX40L, MHC-II, ICAM-1, LFA-1, B7-H2, CD40, CD70, CD48, CD52, TL1A, GITRL, CD30L, TIM1, TIM3, TIM4, SLAM, LIGHT, HVEM, PD-1, CTLA-4, TIGIT, LAG3, OX40, VISG-3, VISG-8, ICOS, CD28, CD40L, DR3, GITR, CD30, CD2, CD226, CD160, BTLA, PD1H, LAIR1, or NKCR2B4. The extracellular vesicle-bound checkpoint protein herein may include the same checkpoint protein in the method provided supra.

### D. Methods of Treating a Subject by Removing or Replacing Checkpoint Proteins/Co-Stimulatory Factors, Extracellular Vesicles, and Markers

As noted above, the present invention is based on the discovery that certain checkpoint proteins or co-stimulatory factors on certain extracellular vesicle (i.e. exosomes) can be used to predict whether a patient will respond to an anti-cancer therapy. This discovery provides evidence that removal and/or replacement of the cancer-promoting checkpoint therapy on said exosomes can be used to treat a patient with cancer and/or be used to increase the efficacy of an anti-cancer medication. In one embodiment, a method of increasing the efficacy of an anti-cancer medication in a patient in need thereof is provided, the method comprising: contacting of a biological sample from the patient with a first reagent, the biological sample comprising a cancer-promoting extracellular vesicle comprising a checkpoint protein and a source-specific marker, the checkpoint protein suppressing an immune response to the cancer, wherein in the contacting, the first reagent binds the cancer-promoting extracellular vesicles, thereby removing the cancer-promoting extracellular vesicles from the biological sample and obtaining a purified biological sample; and introducing the purified biological sample to the patient thereby increasing the efficacy of the anti-cancer medication.

In an embodiment, the first reagent binds the source-specific marker, thereby removing the cancer-promoting extracellular vesicles from the biological sample. In an embodiment, the anti-cancer medication comprises a checkpoint inhibitor therapy comprising an inhibitor of signaling of the checkpoint protein.

Increases and Decreases in the Assessing

As noted above, the first and second biological samples are assessed for relative increases and decreases in the amount of the second checkpoint protein on the extracellular vesicle that has the source-specific marker. In some embodiments, the amount of the second checkpoint protein on the extracellular vesicle that has the source-specific marker of the second biological sample is elevated at least 10%, 20%, 30%, 40%, 50%, 60%, 70%, 80%. 90%, 1 fold, 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 33 fold, 100 fold, 333 fold, 1000 fold, 3333 fold, or 10,000 fold above the amount of the second checkpoint protein on the extracellular vesicle that has the source-specific marker of the first biological sample, and the second checkpoint inhibitor therapy comprising an inhibitor of signaling of the second checkpoint protein. In these embodiments, the second checkpoint protein is optionally an immunosuppressive protein and the second checkpoint inhibitor therapy optionally comprises an inhibitor of signaling of the second checkpoint protein.

In some embodiments, the amount of the second checkpoint protein on the extracellular vesicle that has the source-specific marker of the second biological sample is decreased at least 10%, 20%, 30%, 40%, 50%, 60%, 70%, 80%. 90%, 1 fold, 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 33 fold, 100 fold, 333 fold, 1000 fold, 3333 fold, or 10,000 fold below the amount of the second checkpoint protein on the extracellular vesicle that has the source-specific marker of the first biological sample, and the second checkpoint inhibitor therapy comprising an inhibitor of signaling of the second checkpoint protein. In these embodiments, the second checkpoint protein is optionally an immunostimulatory protein and the method optionally comprises administering a second checkpoint stimulatory therapy to the patient after assessing a first biological sample and a second biological sample. In such an embodiment, the second checkpoint stimulatory therapy optionally comprises administering extracellular vesicles comprising the second checkpoint protein and the source-specific marker.

Administration of Anti-Cancer Medications, Including Checkpoint Inhibitor Therapies

The anti-cancer medication of the present invention can be administered by any suitable route, for example, by injection, by oral, pulmonary, topical or other modes of administration. In general, pharmaceutical compositions of the present invention, comprise, among other things, pharmaceutically acceptable diluents, preservatives, solubilizers, emulsifiers, adjuvants and/or carriers. Such compositions can include diluents of various buffer content (e.g., Tris-HCl, acetate, phosphate), pH and ionic strength; and additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol). The compositions can be incorporated into particulate preparations of polymeric compounds such as polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington's Pharmaceutical Sciences, 18th Ed. (1990, Mack Publishing Co., Easton, Pa. 18042) pages 1435-1712 which are herein incorporated by reference. The pharmaceutical composition of the present invention can be prepared, for example, in liquid form, or can be in dried powder form (e.g., lyophilized).

In yet another embodiment, the anti-cancer medication of the present invention can be delivered in a controlled release system, such as using an intravenous infusion, an implantable osmotic pump, a transdermal patch, liposomes, or other modes of administration. In a particular embodiment, a pump may be used (see Langer, supra; Sefton, CRC Crit. Ref. Biomed. Eng. (1987) 14:201; Buchwald et al., Surgery (1980) 88:507; Saudek et al., N. Engl. J. Med. (1989) 321:574). In another embodiment, polymeric materials may be employed (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. (1983) 23:61; see also Levy et al., Science (1985) 228:190; During et al., Ann. Neurol. (1989) 25:351; Howard et al., J. Neurosurg. (1989) 71: 105). In yet another embodiment, a controlled release system can be placed in proximity of the target tissues of the animal, thus requiring only a fraction of the systemic dose (see, e.g., Goodson, in Medical Applications of Controlled Release, supra, (1984) vol. 2, pp. 115-138). In particular, a controlled release device can be introduced into an animal in proximity to the site of inappropriate immune activation or a tumor. Other controlled release systems are discussed in the review by Langer (Science (1990) 249:1527-1533).

Methods of Measuring the Checkpoint Protein and the Source-Specific Marker and Methods of Capturing Exosomes Comprising the Checkpoint Protein and the Source-Specific Marker

A non-limiting list of methods for measuring the checkpoint protein and source specific markers include immunoblotting (i.e. western blotting), semi-quantifiable and quantifiable immunohistochemistry, semi-quantifiable and quantifiable immunofluorescence, immunolabeling followed by transmission electron microscopy (TEM), NANOSIGHT® nanoparticle tracking systems, reverse phase protein arrays (RPPA), density gradient centrifugation optionally followed by an immunolabeling approach, enzyme-linked immunosorbent assay (“ELISA”), immunoglobin-conjugated bead labelling followed optionally by flow cytometry, ELISPOT, cytometric bead array or other multiplex methods, and other immunoaffinity-based methods. Immunolabelling followed by TEM may include immunogold-labeling with a monoclonal antibody (e.g. against the extracellular domain of PD-L1). An ordinary skilled artisan would be able to identify methods for detecting the levels of particular proteins.

Purification of exosomes may include the use of commercially available kits, the use of polymer based pull-down methods, the use of immunoglobin-conjugated bead pull-down methods, density gradient centrifugation and ultracentrifugation. Without limitation, density gradient centrifugation and ultracentrifugation may include the use of iodixanol, ficoll, sucrose, lithium, and other gradients and may include centrifugal forces between 16,500 G and 100,000 G.

Methods for Removing and Replacing the Exosomes Comprising the Combination of the Cancer-Promoting Checkpoint Proteins and Source-Specific Markers

As noted above, the initial step of measuring the checkpoint protein on a particular exosome involves the isolation of all or part of the exosomes derived from a particular cell type or the isolation of all or part of the exosomes carrying a particular checkpoint protein. In some embodiments, a second step may include the reciprocal isolation step. If the isolation of all or part of the exosomes derived from a particular cell type was the first step, the second step would involve the isolation of all or part of the exosomes carrying a particular checkpoint protein. If the isolation of all or part of the exosomes carrying a particular checkpoint protein was the first step, the second step would involve the isolation of all or part of the exosomes derived from a particular cell type.

These methods may also be used to purify and produce exosomes with anti-cancer properties or exosomes with immunopromoting properties to treat the cancer and/or increase the efficacy of the anti-cancer medication. In some embodiments, the method further comprises purifying, isolating, and/or producing exosomes with said immunopromoting properties and introducing these exosomes into the patient with or without the depletion and/or removal of the cancer-promoting or anti-cancer medication inhibiting exosomes. Methods of purifying and isolating said exosomes are provided supra. Methods for producing exosomes with said immunopromoting properties are known in the art, and can include the production of recombinant checkpoint proteins, recombinant source-specific markers, and the use of synthesized phospholipids and cholesterol to make the exosomes. Alternatively, the introduced exosome may comprise a switch receptor in addition to or in the alternative of a immunostimulating checkpoint protein. Switch receptors and methods of producing switch receptors are described in U.S. Patent Application Publication No.: 2017/0360913, which is incorporated by reference in its entirety herein.

In brief, switch receptors may comprise the extracellular (or extraexosomal) binding domain of an inhibitory signaling molecule (i.e. PD-1, TIGIT, TIM3, CTLA-4, CD160, BTLA) and the extracellular binding domain of stimulatory ligand (i.e. an extracellular binding domain of CD40, OX40L, CD70, B7-H2, TL1A GITRL, CD58, CD48).

In another aspect, the invention includes a method for treating cancer in a patient in need thereof. The method comprises assessing the levels of PD-L1 and/or PD-1 and the levels of CD155 and/or CD112 and/or TIGIT in a biological sample comprising an extracellular vesicle obtained from the patient before administration of a first therapy. When the the level of PD-L1 and/or PD-1 is high and the level of CD155 and/or CD112 and/or TIGIT is low in comparison to a reference sample, the first therapy is administered to the patient. When the levels of PD-L1 and/or PD-1 and the levels of CD155 and/or CD112 and/or TIGIT are high in comparison to a reference sample, a second therapy is administered alone or together with the first therapy to the patient. In certain embodiments, the first therapy comprises an inhibitor of PD-1 or PD-L1 and the second therapy comprises an inhibitor of TGIT. In certain embodiments, the inhibitor is selected from the group consisting of an antibody, a chemical compound, or an siRNA.

In another aspect, the invention includes a method for treating cancer in a patient in need thereof. The method comprises assessing the level of CD155 and/or CD112 in a biological sample comprising an extracellular vesicle obtained from the patient before administration of a first therapy, and assessing the level of CD155 and/or CD112 in a biological sample comprising an extracellular vesicle obtained from the patient after administration of a first therapy. When the the level of CD155 and/or CD112 in the second biological sample is increased in comparison to the first biological sample, the patient is determined to not be responding to the first therapy, and a second therapy is administered to the patient. In certain embodiments, the first therapy comprises an inhibitor of PD-1 or PD-L1 and the second therapy comprises an inhibitor of TGIT. In certain embodiments, the inhibitor is selected from the group consisting of an antibody, a chemical compound, or an siRNA.

In another aspect, the invention includes a method for treating cancer in a patient in need thereof. The method comprises assessing the level of a co-stimulatory factor in a first biological sample comprising an extracellular vesicle obtained from the patient before administration of a therapy, and assessing the level of the co-stimulatory factor in a second biological sample comprising an extracellular vesicle obtained from the patient after administration of a therapy. When the level of the co-stimulatory factor is increased in the second biological sample in comparison to the first biological sample, the patient is determined to be responding to therapy, and the therapy is continued. In certain embodiments, the method further comprises assessing the level of the co-stimulatory factor in a third biological sample comprising an extracellular vesicle obtained from the patient. When the level of the co-stimulatory factor is decreased in the third biological sample in comparison to the first biological sample, the patient is determined to be responding to therapy, and the therapy is continued. When the level of the co-stimulatory factor is increased in the third biological sample in comparison to the first biological sample, the patient is determined to not be responding to therapy, and the therapy is discontinued and a alternative therapy is administered to the patient. In certain embodiments, the second biological sample is obtained three weeks after the first biological sample. In certain embodiments, the third biological sample is obtained 2, 3, 4, 5, 6, 7, 8, or 9 weeks after the second biological sample. In certain embodiments, the co-stimulatory factor is selected from the group consisting of CD40, CD40L, OX40, OX40L, CD137, and CD137L.

In another aspect, the invention includes a method for treating cancer in a patient in need thereof. The method comprises assessing the level of phosphorylated HRS in a biological sample comprising tumor tissues or an extracellular vesicle obtained from the patient. When the level of phosphorylated HRS is high in comparison to a reference sample, the patient is administered a combination therapy. In certain embodiments, the combination therapy comprises a drug that inhibits PD-1 or PD-L1 and a drug that inhibits the MAPK pathway.

In certain embodiments, the cancer to be treated is a melanoma.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

Purification of the Exosomes

For exosome purification from cell culture supernatants, cells were cultured in media supplemented with 10% exosome-depleted FBS. Bovine exosomes were depleted by overnight centrifugation at 100,000 g. Supernatants were collected from 48-72 hr. cell cultures and exosomes were purified by a standard differential centrifugation protocol (8-10). Briefly, culture supernatants were centrifuged at 2,000 g for 20 min to remove cell debris and dead cells (Beckman Coulter, Allegra X-14R). Supernatants were then centrifuged at 16,500 g for 45 min (Beckman Coulter, J2-HS) to remove larger vesicles. Exosomes were pelleted by ultracentrifugation of the supernatants at 100,000 g for 2 hr. at 4° C. (Beckman Coulter, Optima XPN-100). The pelleted exosomes were suspended in PBS and collected by ultracentrifugation at 100,000 g for 2 hr. at 4° C.

For purification of circulating exosomes by differential centrifugation, venous citrated blood from melanoma patients or healthy donors was centrifuged at 1,550 g for 30 min to obtain cell-free plasma (Beckman Coulter, Allegra X-14R). Then 1 ml of the obtained plasma was centrifuged at 16,500 g for 45 min to remove larger vesicles (Eppendorf, 5418R). The collected supernatant was centrifuged at 100,000 g for 2 hr. at 4° C. (Beckman Coulter, Optima™ MAX-XP) to pellet the exosomes. The exosome pellet was suspended in PBS and collected by ultracentrifugation at 100,000 g for 2 hr. at 4° C.

For purification of circulating exosomes using the exosome isolation kit, 100 μL of cell-free plasma were first pre-cleared of large membrane vesicles by centrifugation at 16,500 g for 45 min (Eppendorf, 5418R). Exosomes were then purified from the supernatants using the exosome isolation kit (Invitrogen, Cat #4484450).

Characterization of the Purified Exosomes by Flow Cytometry

For characterization with flow cytometry, purified exosomes (25 μg) were incubated with 20 μL CD63-coated magnetic beads (Invitrogen, Cat #10606D) in 100 μL PBS with 0.1% bovine serum albumin (BSA) overnight at 4° C. with mixing. Exosomes-coated beads were then washed, and incubated with fluorophore-labeled antibodies, followed by analysis on a LSR II flow cytometer (BD Biosciences).

Characterization of the Purified Exosomes by Transmission Electron Microscopy

For verification of purified exosomes using electron microscopy, purified exosomes suspended in PBS were dropped on formvar carbon coated nickel grids. After staining with 2% uranyl acetate, grids were air-dried and visualized using a JEM-1011 transmission electron microscope. For immunogold labeling, purified exosomes suspended in PBS were placed on formvar carbon coated nickel grids, blocked, and incubated with the mouse anti-human monoclonal antibody that recognizes the extracellular domain of PD-L1 (clone 5H1-A3) (Dong et al., (2002) Nat. Med. 8, 793-800), followed by incubation with the anti-mouse secondary antibody conjugated with protein A-gold particles (5 nm). Each staining step was followed by five PBS washes and ten dH₂O washes before contrast staining with 2% uranyl acetate.

Characterization of the Purified Exosomes by NANOSIGHT®

The size of exosomes was determined using NANOSIGHT® NS300 (Malvern Instruments), which is equipped with fast video capture and particle-tracking software.

Characterization of the Purified Exosomes by Density-Gradient Centrifugation

For iodixanol density-gradient centrifugation, exosomes harvested by differential centrifugation was loaded on top of a discontinuous iodixanol gradient (5%, 10%, 20% and 40% made by diluting 60% OptiPrep™ aqueous iodixanol with 0.25 M sucrose in 10 mM Tris) and centrifuged at 100,000 g for 18 hr. at 4° C. Twelve fractions with equal volumes were collected from the top of the gradients, with the exosomes distributed at the density range between 1.13 to 1.19 g/ml, as previously demonstrated (Thery et al., (2006) Curr. Protoc. Cell Biol. 30, 3.22.1-3.22.29; Colombo et al, (2014) Annu. Rev. Cell Dev. Biol. 30, 255-289; Tibes et al. (2006) Mol. Cancer Ther. 5, 2512-2521). The exosomes were further pelleted by ultracentrifugation at 100,000 g for 2 hr. at 4° C.

Immunoprecipitation of PD-L1 Proteins from the Cell Culture Supernatants

To analyze PD-L1 proteins secreted by melanoma cells, WM9 cells were treated with IFN-7 (100 ng/ml) in FBS-depleted RPMI 1640 medium for 48 hr. The culture supernatants were collected and centrifuged at 2,000 g for 20 min, and then at 16,500 g for 45 min. For further exosome isolation, supernatants were subjected to ultracentrifugation at 100,000 g for 2 hr. at 4° C. For immunoprecipitation of PD-L1 proteins, the concentrated supernatants were incubated with 5 μg/ml mouse anti-human 5H1-A3 monoclonal antibodies and protein A/G agarose overnight at 4° C. The immunoprecipitated proteins were loaded on 12% SDS-polyacrylamide gel electrophoresis and transferred onto nitrocellulose membranes. The blots were blocked with 5% non-fat milk at room temperature for 1 hr., and incubated overnight at 4° C. with two different rabbit anti-human monoclonal antibodies targeting the intracellular domain (Cell Signaling Technology, Cat #13684) and extracellular domain (Cell Signaling Technology, Cat #15165) of PD-L1, respectively, followed by incubation with the horseradish peroxidase (HRP)-conjugated anti-rabbit secondary antibody (Cell Signaling Technology, Cat #7074) at room temperature for 1 nr. The blots were developed with ECL detection reagents (Pierce).

ELISA

For detection of exosomal PD-L1, ELISA plates (96-well) (Biolegend) were coated with 0.25 μg per well (100 μL) of monoclonal antibody against PD-L1 (clone 5H1-A3) overnight at 4° C. Free binding sites were blocked with 200 μL of blocking buffer (Pierce) for 1 hr. at room temperature. Then 100 μL of exosome samples, either purified from plasma or cell culture supernatants were added to each well and incubated overnight at 4° C.

Biotinylated monoclonal PD-L1 antibody (clone MIH1, eBioscience) was added and incubated for 1 hr. at room temperature. A total of 100 μL per well of horseradish peroxidase-conjugated streptavidin (BD Biosciences) diluted in PBS containing 0.1% BSA was then added and incubated for 1 hr. at room temperature. Plates were developed with tetramethylbenzidine (Pierce) and stopped with 0.5N H2SO4. The plates were read at 450 nm with a BioTek plate reader. Recombinant human PD-L1 protein (R&D Systems, Cat #156-B7) was used to make the standard curve. Recombinant P-selectin protein (R&D Systems, Cat #137-PS) and mouse PD-L1 protein (R&D Systems, Cat #1019-B7) were used as negative controls to verify the detection specificity. The result of standard curve demonstrated that the established ELISA exhibited a reliable linear detection range from 0.2 to 12 ng/ml.

For detection of exosomal PD-L1 or other checkpoints proteins on exosomes with source-specific or non-source specific markers, ELISA plates (96-well) (Biolegend) were coated with 0.25 μg per well (100 μL) of antibodies against source specific or non-specific markers overnight at 4° C. In some embodiments, the marker comprises: CD63/CD9/CD81, CD163, CD11b, CD11c, CD115, Gr-1, CD8, CD4, CD44, CD90, CD105, SFA, EGFR, VEGFR, PD-L1, CD155, CD112, B7-1, B7-2, B7-H3, Galectin-9, Siglec-15, OX40L, MHC-II, ICAM-1, LFA-1, B7-H2, CD40, CD70, CD48, CD52, CD137, TL1A, GITRL, CD30L, TIM1, TIM3, TIM4, SLAM, LIGHT, HVEM, PD-1, CTLA-4, TIGIT, PVRIG, LAG3, OX40, VISG-3, VSIG-8, ICOS, CD28, CD27, CD40L, DR3, GITR, CD30, CD2, CD226, B7-H1, CD160, BTLA, PD1H, LAIR1, 2B4. Free binding sites were blocked with 200 μL of blocking buffer (Pierce) for 1 hr. at room temperature. Then 40 μL of biological samples, either plasma or other body fluids were added to each well and incubated overnight at 4° C.

Biotinylated antibodies targeting to the checkpoints or other markers was added and incubated for 1 hr. at room temperature. In some embodiments, the marker comprises: PD-L1, CD155, CD112, B7-1, B7-2, B7-H3, Galectin-9, Siglec-15, OX40L, MHC-II, ICAM-1, LFA-1, B7-H2, CD40, CD70, CD48, CD52, CD137, TL1A, GITRL, CD30L, TIM1, TIM3, TIM4, SLAM, LIGHT, HVEM, PD-1, CTLA-4, TIGIT, PVRIG, LAG3, OX40, VISG-3, VSIG-8, ICOS, CD28, CD27, CD40L, DR3, GITR, CD30, CD2, CD226, B7-H1, CD160, BTLA, PD1H, LAIR1, 2B4. A total of 100 μL per well of horseradish peroxidase-conjugated streptavidin (BD Biosciences) diluted in PBS containing 0.1% BSA was then added and incubated for 1 hr. at room temperature. Plates were developed with tetramethylbenzidine (Pierce) and stopped with 0.5N H2SO4. The plates were read at 450 nm with a BioTek plate reader.

PD-1-PD-L1 Binding Assay

To test the binding of exosomal PD-L1 to PD-1, 100 μL of exosome samples of different concentrations were captured onto PD-L1 antibody (clone 5H1-A3)-coated 96-well ELISA plates by overnight incubation at 4° C. Then 100 μL of 4 μg/ml biotin-labeled PD-1 protein (BPS Bioscience, Cat #71109) was added and incubated for 2 hr. at room temperature. A total of 100 L per well of horseradish peroxidase-conjugated streptavidin (BD Biosciences) diluted in PBS containing 0.1% BSA was then added and incubated for 1 hr. at room temperature. Plates were developed with tetramethylbenzidine (Pierce) and stopped using 0.5N H2SO4. The plates were read at 450 nm with a BioTek plate reader. Recombinant human PD-L1 protein directly coated onto the plates was used as the positive control.

Other Methods

Other methods of detecting exosomes, checkpoint proteins, and markers, including source-specific markers are provided in WO 2019/094692, published May 16, 2019, which is incorporated herein by reference in its entirety.

### Example 1

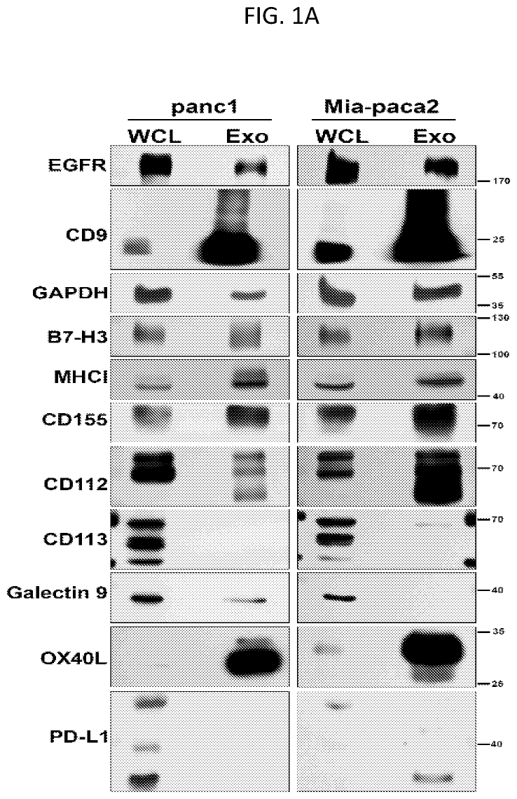

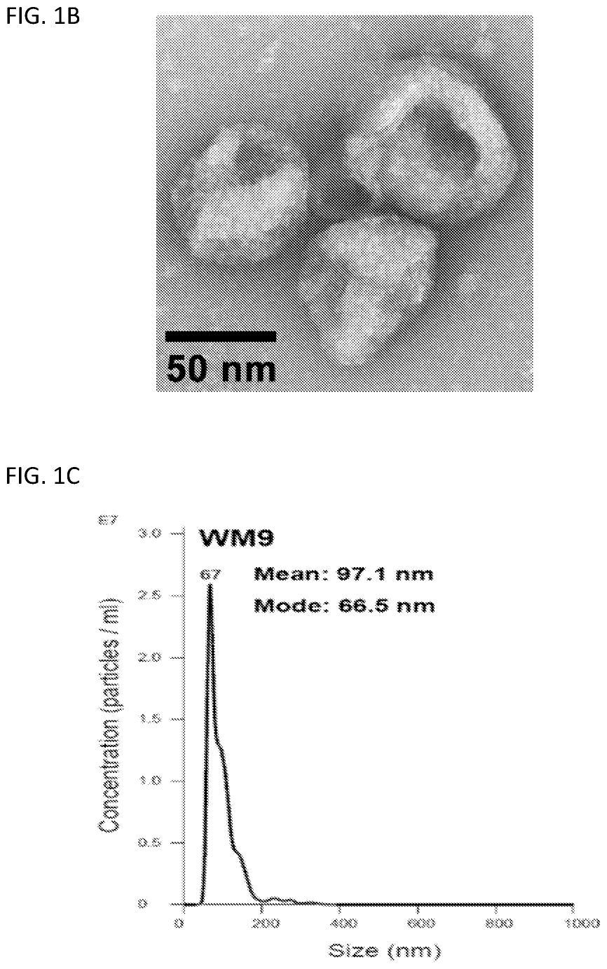

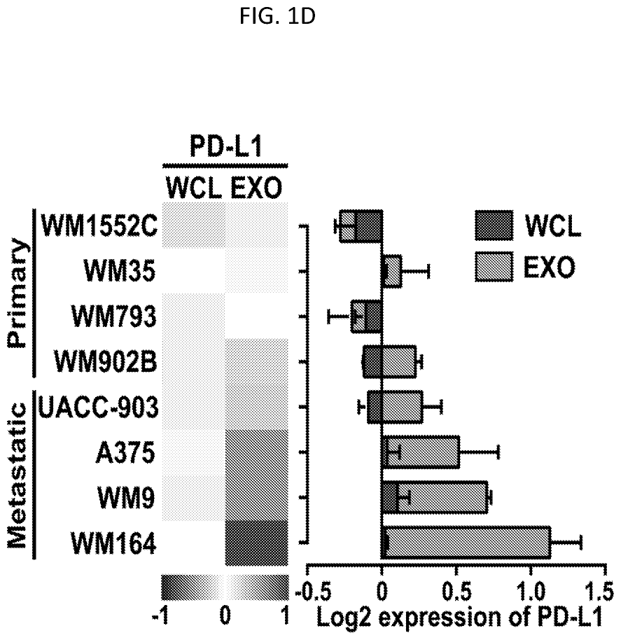

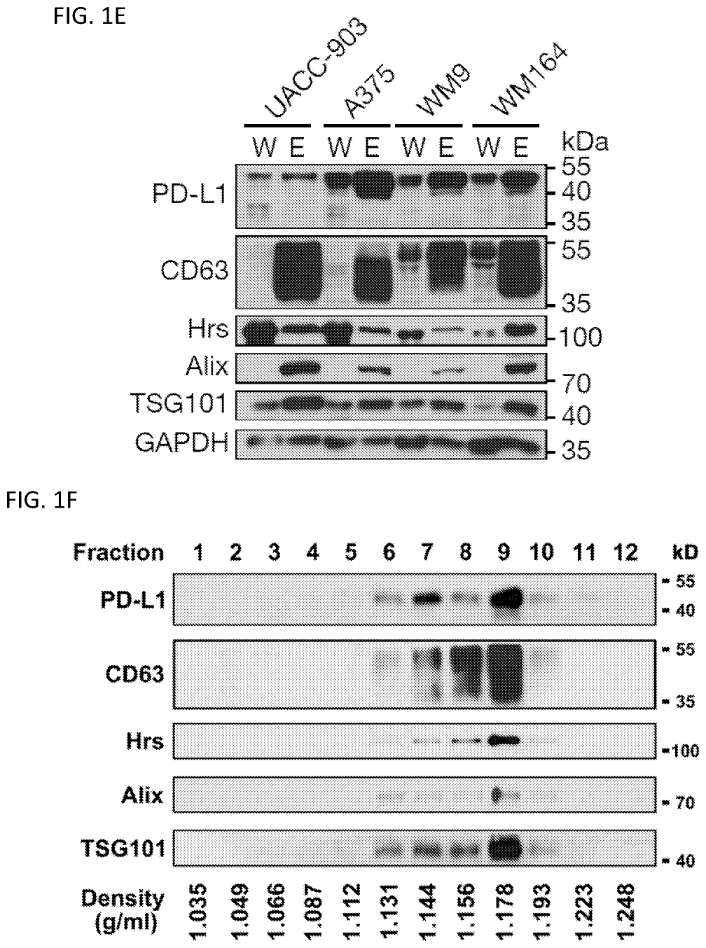

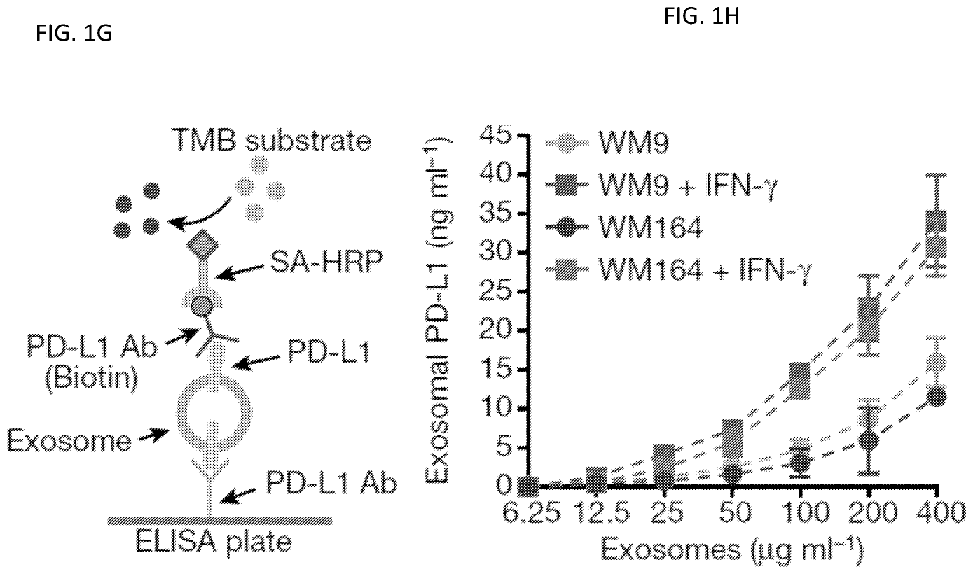

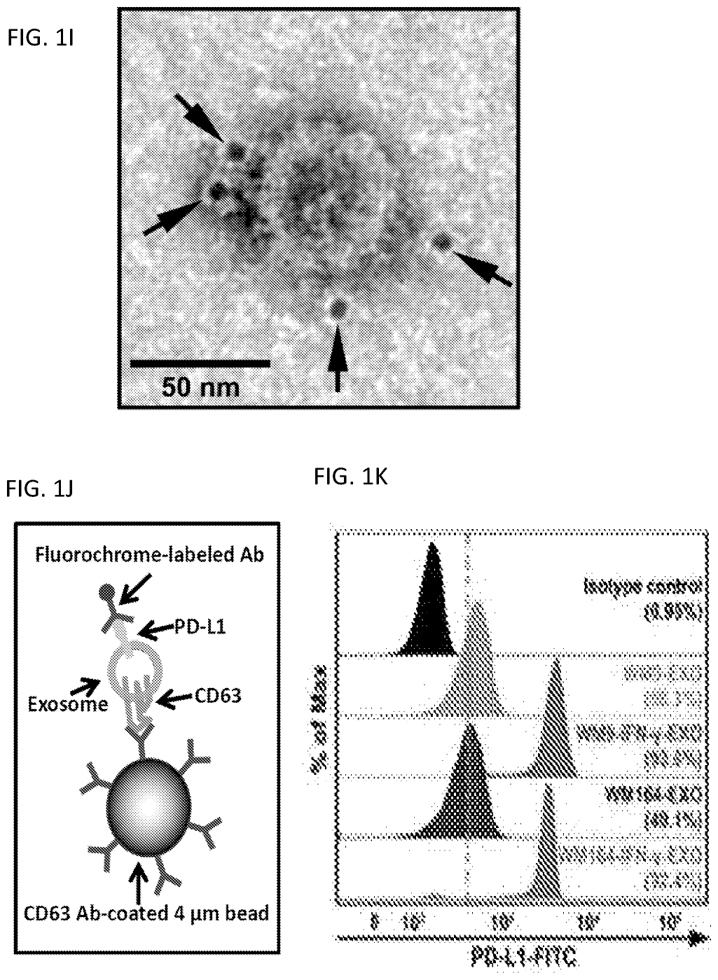

Immunoblots of checkpoint proteins (e.g. CD155, CD112, CD113, Galectin 9, OX40L, PD-L1) and source-specific proteins (e.g. EGFR, CD9, GAPDH, B7-H3, MHC-I) in exosomes (“EXO”) and whole cell lysates (“WCL”) derived from pancreatic cancer cells lines PANC-1 and Mia-PaCa2 were performed (FIG. 1A). B7-H3, CD155, and OX40L were enriched in the exosomes compared to the whole cell lysates; however PD-L1 levels were almost non-detectable in the lysates, indicating that pancreatic cancer cells might not be the major source of certain checkpoints bound to exosomes detected in patient's blood and serum.

### Example 2

Transmission electron microscopy was performed on exosomes purified from culture supernatant of human melanoma cells, demonstrating proof of concept for single exosome imaging. A representative image is depicted in FIG. 1B.

### Example 3

Characterization of purified exosomes was performed using NANOSIGHT® nanoparticle tracking system. A representative size curve is depicted in FIG. 1C.

### Example 4

Reverse phase protein array (RPPA) of the levels of PD-L1 in whole cell lysate (“WCL”) and exosomes (“EXO”) secreted by primary or metastatic melanoma cell lines was performed (FIG. 1D). A heatmap of reverse phase protein array (RPPA) was generated and depicts that PD-L1 is elevated in exosomes of A375, WM9, and WM164 cancer cell lines and WM902B primary cell lines (FIG. 1D, left panel). The Log 2 transformed RPPA data is shown in FIG. 1D, right panel.

### Example 5

Immunoblots for PD-L1 in the whole cell lysate (“W”) and purified exosomes (“E”) were performed and the results are depicted in FIG. 1E. The same amounts of whole cell lysates and exosome proteins were loaded. CD63, Hrs, Alix, and TSG101 were used as exosome markers. GAPDH was used as the loading control. The exosomes were enriched with CD63 and exosomes from the A375, WM9, and WM164 cancer cell lines were significantly enriched with PD-L1, thus demonstrating that the results with RPPA and immunoblot approaches corresponded with each other.

### Example 6

Density gradient centrifugation confirmed that PD-L1 secreted by metastatic melanoma cells co-fractionated with exosome markers CD63, Hrs, Alix, and TSG101 as in Example 4. The results are depicted in FIG. 1F.

### Example 7

PD-L1 on the surface of exosomes secreted by human cells was determined by ELISA using monoclonal antibodies against the extracellular domain of PD-L1 (FIG. 1G). The amount of PD-L1 on exosomes as the concentration of exosomes increases and as the cell lines are treated with interferon-gamma is depicted in FIG. 1H.

### Example 8

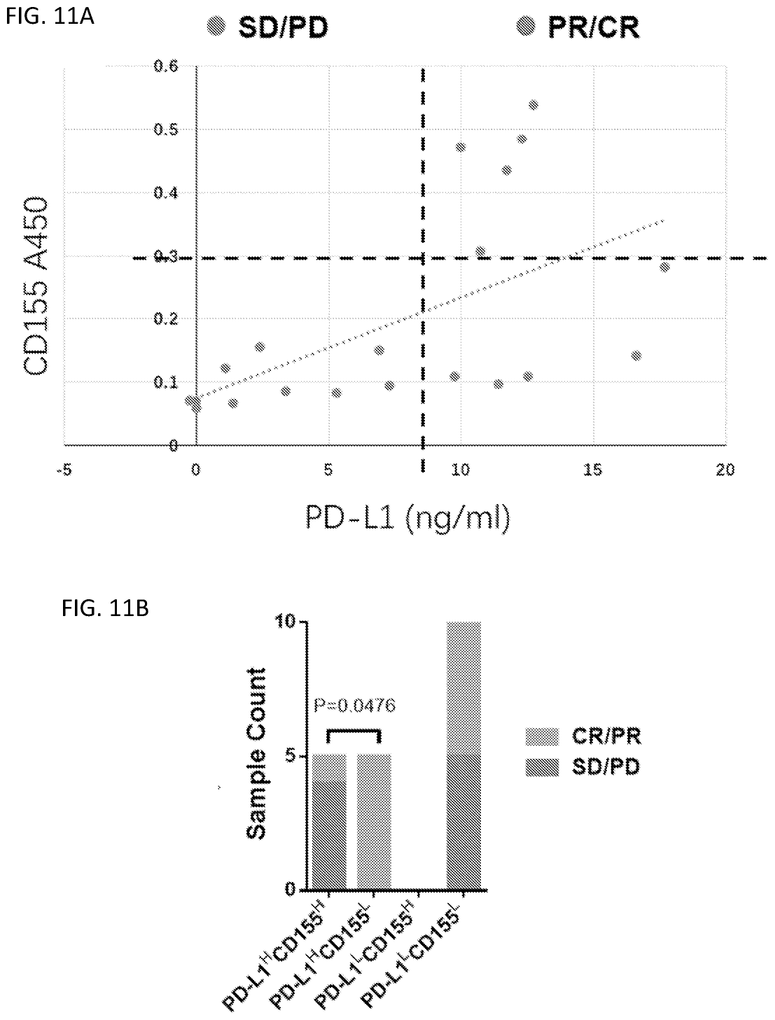

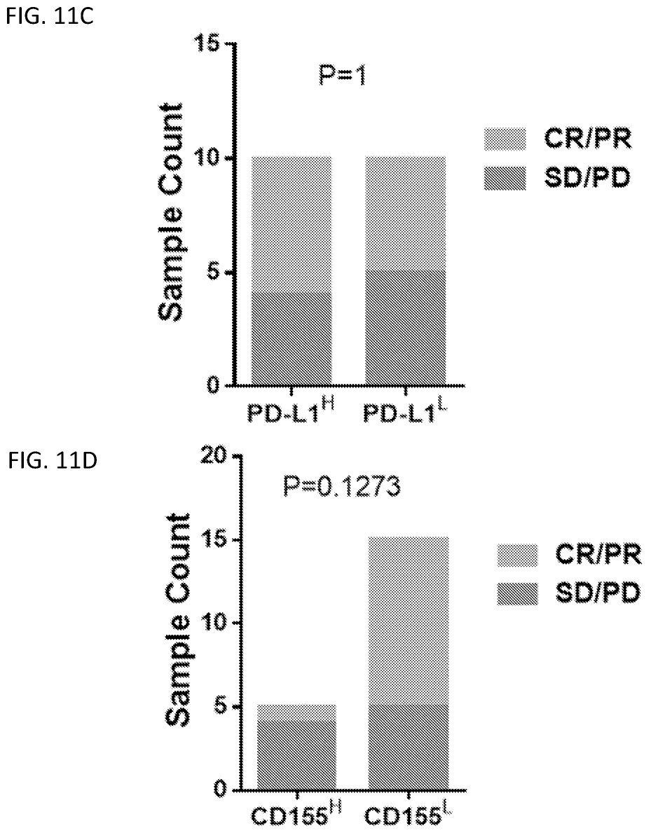

Transmission electron microscopy and immune-gold labeling of PD-L1 was performed. A representative TEM image of cell-derived exosomes immunogold-labeled with a monoclonal antibody against the extracellular domain of PD-L1 is depicted in FIG. 11, with the arrowheads indicating the 5-nm gold particles.

### Example 9

Flow cytometric analysis was performed on the same exosomes from Example 6. PD-L1 protein was labelled with a FITC conjugated antibody and CD63 positive exosomes were pulled down and identified with anti-CD63 antibody conjugated 4 μm beads. FIG. 1J depicts a diagram of flow cytometric analysis of exosomal PD-L1 by CD63-coated beads. FIG. 1K depicts the secretion of PD-L1 protein on exosome surface by human melanoma cells as determined by flow cytometry, indicating the percentage of beads with PD-L1 exosomes from a representative experiment.

### Example 10

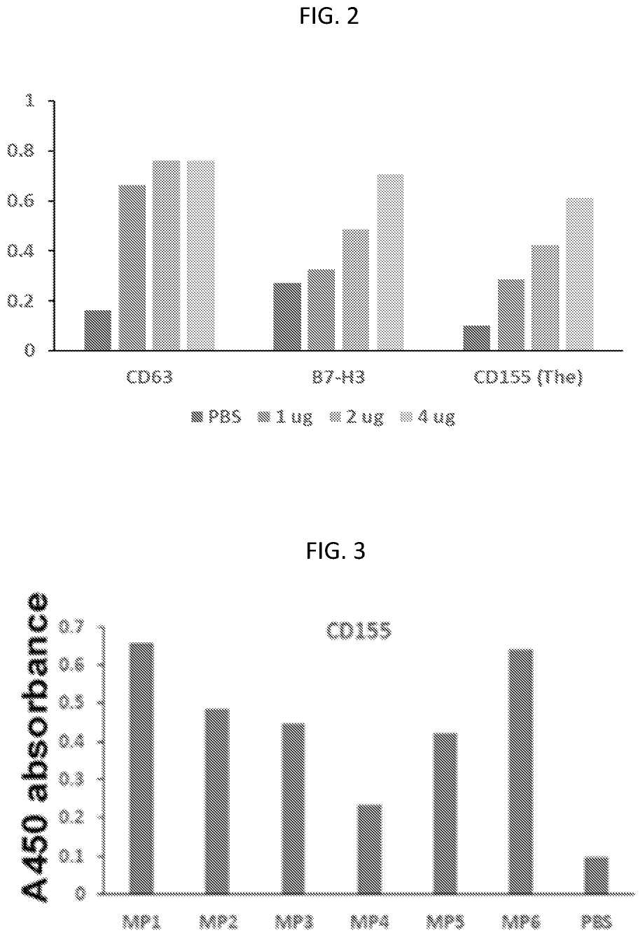

Total exosomes derived from the Panc-1 pancreatic cancer cell line were captured by combination of antibodies against CD63, CD9, and CD81. Biotin labeled anti-CD63, anti-B7-H3, and anti-CD155 antibodies were used to detect the proteins on exosomes by an immunoblot. The exosomes were load with the does 1, 2 and 4 ug to show the sensitivity of the detection assay. FIG. 2 depicts the results measuring immunomodulators including checkpoint proteins on total exosomes.

### Example 11

An enzyme-linked immunosorbent assay (“ELISA”) detecting CD155 protein obtained from exosomes derived from macrophages, and in particular exosomes captured with an anti-CD163 antibody, was performed from blood samples from 6 melanoma patients (individually labelled “MP1” through “MP6”). FIG. 3 depicts the results of the ELISA.

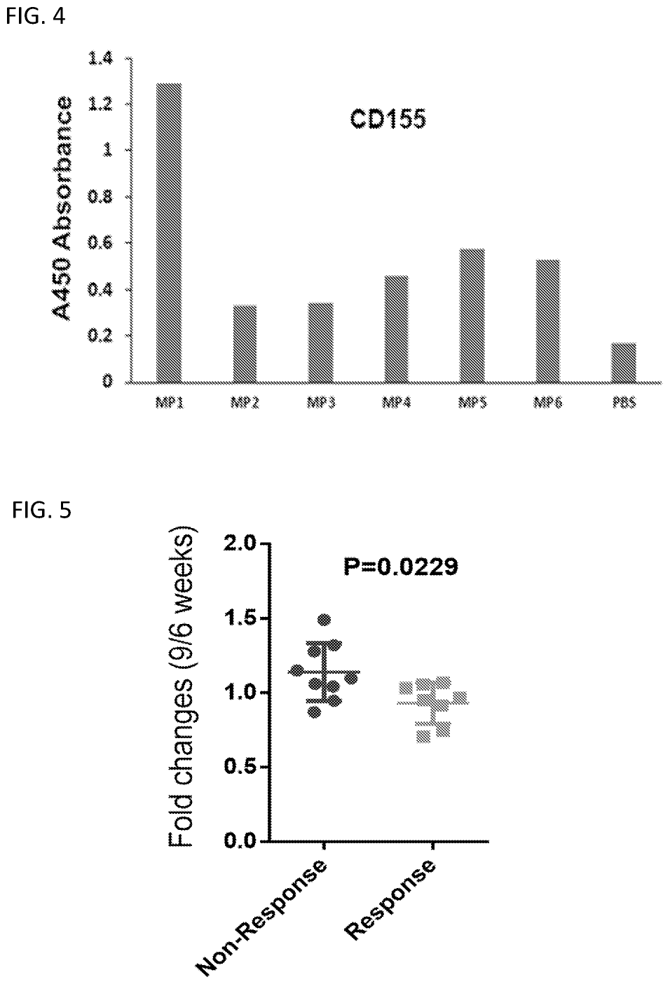

An enzyme-linked immunosorbent assay (“ELISA”) detecting CD155 protein obtained from exosomes captured with an anti-PD-L1 antibody was performed from blood samples from the MP1 through MP6 melanoma patients. FIG. 4 depicts the results of the ELISA.

Without further analysis weighing the contributions of the PD-L1 marker or the source-specific markers indicating the cell type from which the exosomes were derived, it is difficult to predict whether an individual patient will respond to a checkpoint inhibitor therapy, such as an anti-PD-1 antibody. Alternatively, without further temporal information, such as CD155 and CD163 levels, before and after treatment with a checkpoint inhibitor therapy, it is difficult to predict whether an individual patient will respond to the therapy.

### Example 12

CD155 protein levels on circulating exosomes captured with an anti-CD11b antibody were quantified in 8 patients who responded and 9 patients who did not respond to PD-1 inhibitor therapy. Samples were taken throughout the course of the patients' 9 week therapy. The CD155 protein levels on CD11b-positive exosomes from week 9 were divided by the CD155 protein levels on CD11b-positive exosomes from week 6 and the results thereof are depicted in FIG. 5. The patients who failed to respond to PD-1 inhibitor therapy had a significant increase in CD155 protein on circulating CD11b positive exosomes during week 9 over that of week 6 than the same measures from patients who responded to therapy (t-test, p=0.0229). Without wishing to be bound by a particular theory, it is believed that because CD155 is an immunosuppressive checkpoint protein, its elevated presence on CD11b-positive exosomes or macrophage exosomes may serve to bypass the loss of PD-1 signaling by the PD-1 inhibitor therapy resulting in a failure to respond to PD-1 inhibitor therapy.

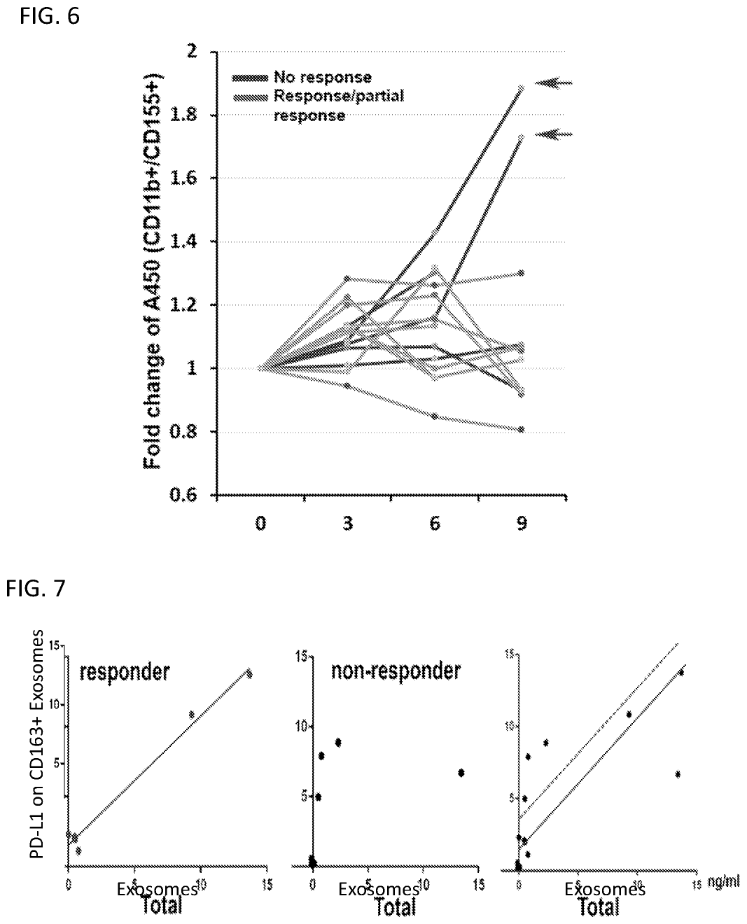

FIG. 6 depicts the quantification of the same measures from the same patients but includes not only the quantifications from blood samples taken week 9 but also the quantifications of blood samples taken weeks 0, 3, and 6 of the study. “Week 0” depicts blood samples taken from patients before the anti-PD-1 antibody therapy. The patients who failed to respond to PD-1 inhibitor therapy also had significant increases in CD155 protein on circulating CD11b positive exosomes at around 6 and 9 weeks treatment than that of patients who responded to therapy (arrows). Without wishing to be bound by a particular theory, it is believed that because CD155 is an immunosuppressive checkpoint protein, elevations in CD155 on CD11b-positive exosomes or macrophage exosomes during an anti-cancer therapy may cause short-lived responses and/or non-responses to therapy because CD155 may serve to bypass the loss of signaling of another checkpoint protein by the checkpoint inhibitor therapy.

### Example 13

The levels of PD-L1 on CD163 positive exosomes relative to total exosomes were quantified for 6 patients who responded and 7 patients who failed to respond to an anti-PD-1 antibody therapy. FIG. 7 depicts the correlation of the levels of total exosomes and the levels of PD-L1 on CD163-positive exosomes in patients who responded to PD-1 inhibitor therapy (left), whereas there was no correlation in patients who failed to respond to PD-1 inhibitor therapy (middle). Accordingly, it can be predicted whether a patient will fail to respond to PD-1 inhibitor therapy, other checkpoint therapies, or other anti-cancer therapies when that patient has levels of checkpoint protein that significantly deviate from the correlation and regression derived from the patients who responded to said checkpoint inhibitor therapy or said anti-cancer therapy. A significant deviation can be determined by placing thresholds off of the regression line based on statistical measures such as: 1) the standard deviation of the distance orthogonal to the line for each responding individual, or from 2) the adjusted value for each responder and non-responder, wherein the adjusted value is determined from the regression analysis from the responders or the whole population of responders and non-responders. For simplicity, a two-dimensional correlation and graphical representation is presented but multi-variate correlation analysis can be used to predict whether a patient will respond to treatment, with each variate being the levels of a different checkpoint protein on exosomes having different markers or source specific markers. The multivariate analysis can also consider other variates that are not based on checkpoint proteins, markers or source-specific markers, or measures of and from exosomes. The presence of a correlation in the responders can be used as part of an algorithm to test whether a particular checkpoint protein or immunostimulator protein on an exosome with a particular source-specific marker or an exosome derived from a particular cell type can be used as a test for predicting whether a patient will respond to a particular treatment. For example, the presence of the correlation of the levels of total exosomes and the levels of PD-L1 on CD163-positive exosomes was a threshold before determining whether the same levels from non-responding patients fell outside of the cut-offs determined from said correlation.

### Example 14

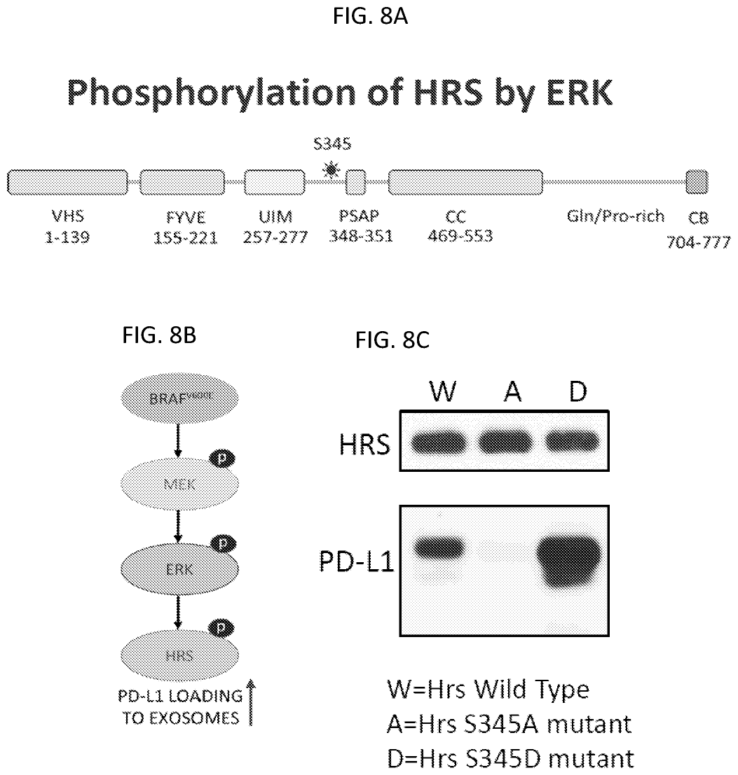

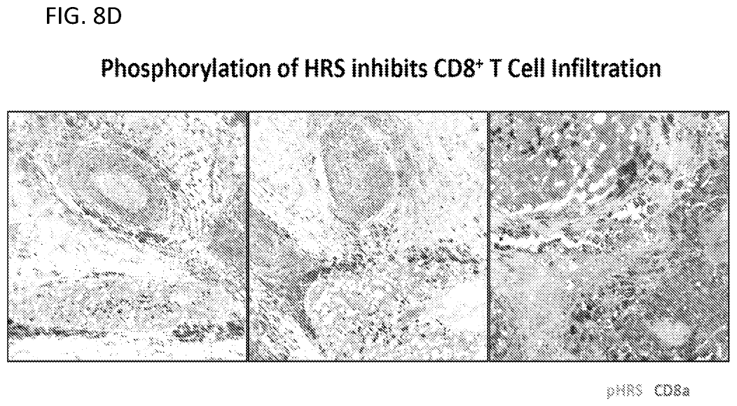

The phosphorylation of HRS by ERK promotes the loading of checkpoint proteins bound to exosomes that have particular source-specific markers. FIG. 8A depicts the phosphorylation site of serine 345 on HRS. FIG. 8B depicts how MAPK signaling in tumors trigger HRS phosphorylation. In melanoma, Braf mutation induced ERK activation can lead to HRS phosphorylation. HRS phosphorylation can also be induced by GPCR, RTK, Ras mutation and TGF-P signaling in different tumors. FIG. 8C depicts substitution studies of serine 345 with alanine (phosphor-defect) or glutamic acid (phosphor-mimetic). The level of PD-L1 was reduced in exosomes from the cells expressing phosphor-defect mutant HRS S345A, and there is more PD-L1 enriched in exosomes derived from cells expressing phosphor-mimetic mutant HRS S345D. FIG. 8D: Immunohistochemistry analysis of melanoma patient samples shows that CD8 positive T cells failed to infiltrate into the tumor when HRS phosphorylation level in tumor cells is high. High levels of HRS phosphorylation blocks CD8 T cell infiltration and diminishes patient response to checkpoints inhibitors.

### Example 15

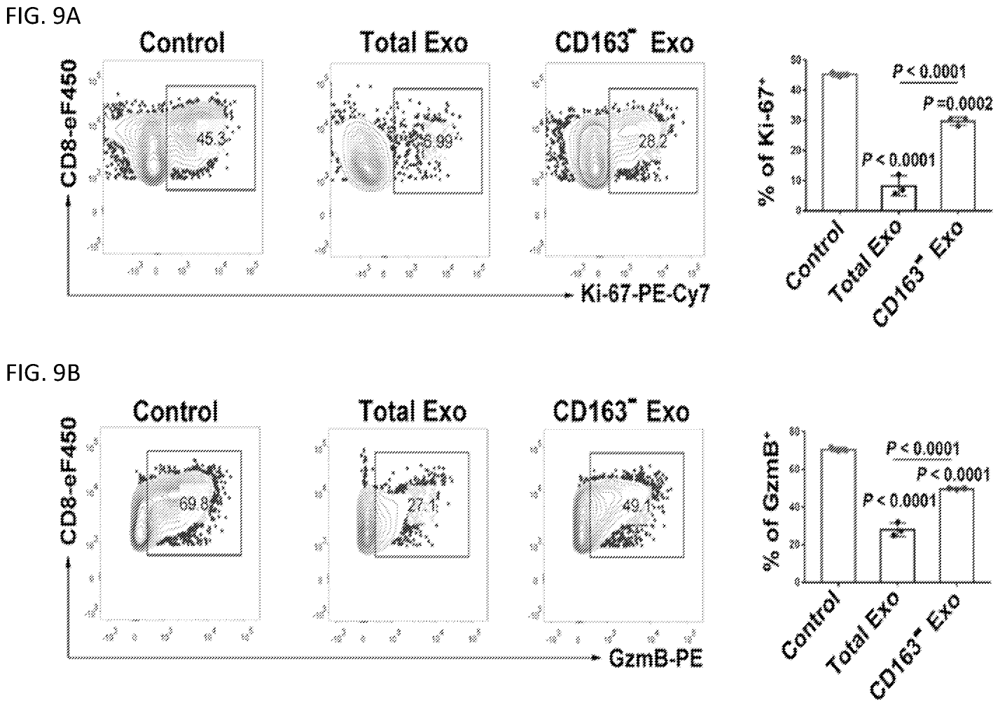

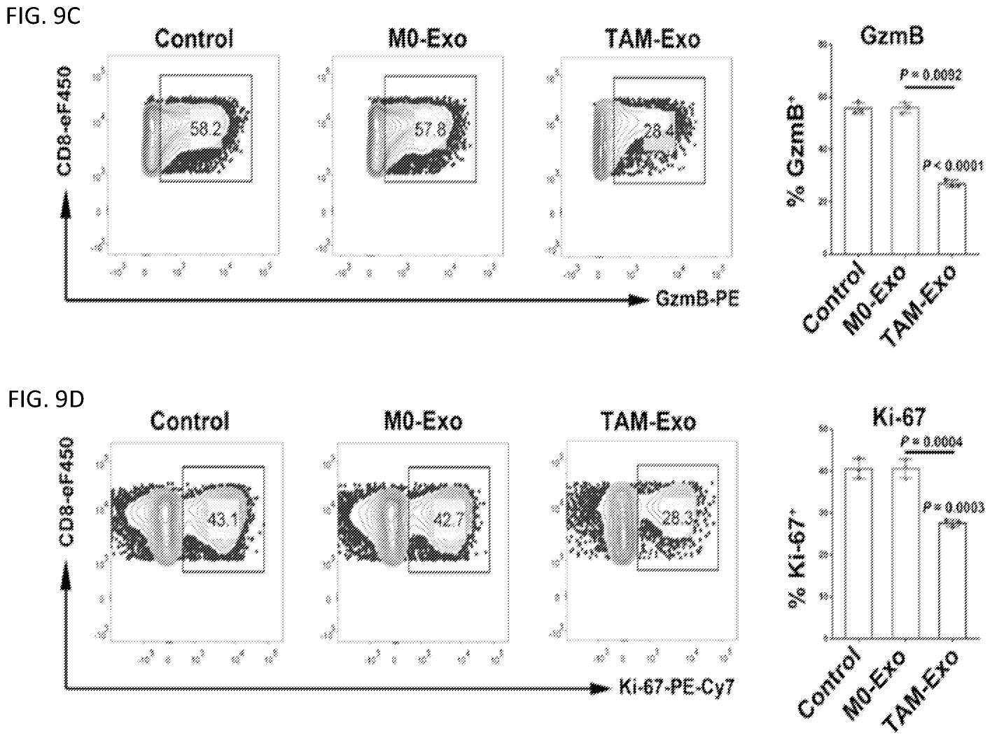

FIGS. 9A-9D depict the experimental results demonstrating the contribution of TAM exosomes to immune suppression in patient tumor tissues using flow cytometry. Tumors (˜1 g) were harvested, washed in PBS, and minced in PBS containing 2 mM EDTA. Tumors were then digested with collagenase IV (0.2 mg/ml, Life Technologies), dispase (2 mg/ml, Life Technologies) and DNase I 0.002 mg/ml, Life Technologies) in RPMI-1640 medium for 20 min at 37° C. Tumor cell suspensions were then cooled down on ice and protease inhibitors (cOmplete™, Roche) were added in a final volume of 45 ml. Tumor tissue exosomes were isolated from tumor-cell suspensions using sequential ultracentrifugation. Briefly, supernatants were centrifuged at 500×g for 5 min, 2000×g for 20 min to remove dead cells and debris, and ultracentrifuged at 10,000×g for 40 min at 4° C. to remove large vesicles. Supernatants were then transferred to new tubes and ultracentrifuged at 100,000×g for 70 min at 4° C. to collect exosomes. Exosomes pellets were washed in 35 mL of PBS and ultracentrifuged again at 100,000×g for 70 min at 4° C. The resulting exosomes preparations were resuspended in PBS and either used immediately or stored at −80° C.

To remove tumor-associated macrophages (TAM)-derived exosomes, 500 μl tumor tissue exosomes (1 g/l) were incubated with Biotin-CD163 Ab labeled-magnetic beads overnight at 4° C. The tube was placed on the magnet for 1 min and supernatant removed to an appropriate tube to obtain tumor tissue exosomes with the depletion of TAM-derived exosomes. Magnetic beads were washed by adding 500 μL cool PBS and mixed well. The tube was placed on the magnet for 1 min and the supernatant discarded. Tumor-associated macrophages (TAM)-derived exosomes from step 10 were finally resuspended in 50-100 μL PBS for subsequent analyses.

FIG. 9A depicts the percent of proliferating cells labelled with Ki-67 after exposure to total exosomes or exosomes without CD163. Exposure to total exosomes or CD163 negative exosomes significantly decreased the percent of CD8 positive cells that are Ki-67 positive and therefore proliferating; and total exosome exposure further decreased the percent of CD8 positive cells that are Ki-67 positive and therefore proliferating compared to that with CD163 negative exosome exposure. FIG. 9B depicts the percent of cells labelled with granzyme B after exposure to total exosomes or exosomes without CD163. Exposure to total exosomes or CD163 negative exosomes significantly decreased the percent of CD8 positive cells that are granzyme B positive; and total exosome exposure further decreased the percent of CD8 positive cells that are granzyme B positive compared to that with CD163 negative exosome exposure. FIG. 9C depicts the percent of proliferating cells labelled with granzyme B after exposure to total exosomes from M0 macrophages (“M0”) and tumor associated macrophages (“TAM”). Exposure to exosomes from resting macrophages failed to decrease the percent of CD8 positive cells that are granzyme B positive. Exposure to exosomes from TAMs significantly decreased the percent of CD8 positive cells that are granzyme B positive compared to both M0 and control conditions. FIG. 9D depicts the percent of proliferating cells labelled with Ki-67 after exposure to total exosomes from M0 macrophages (“M0”) and tumor associated macrophages (“TAM”). Exposure to exosomes from resting macrophages failed to decrease the percent of CD8 positive cells that are Ki-67 positive, and therefore proliferating. Exposure to exosomes from TAMs significantly decreased the percent of CD8 positive cells that are Ki-67 positive compared to both M0 and control conditions.

### Example 16

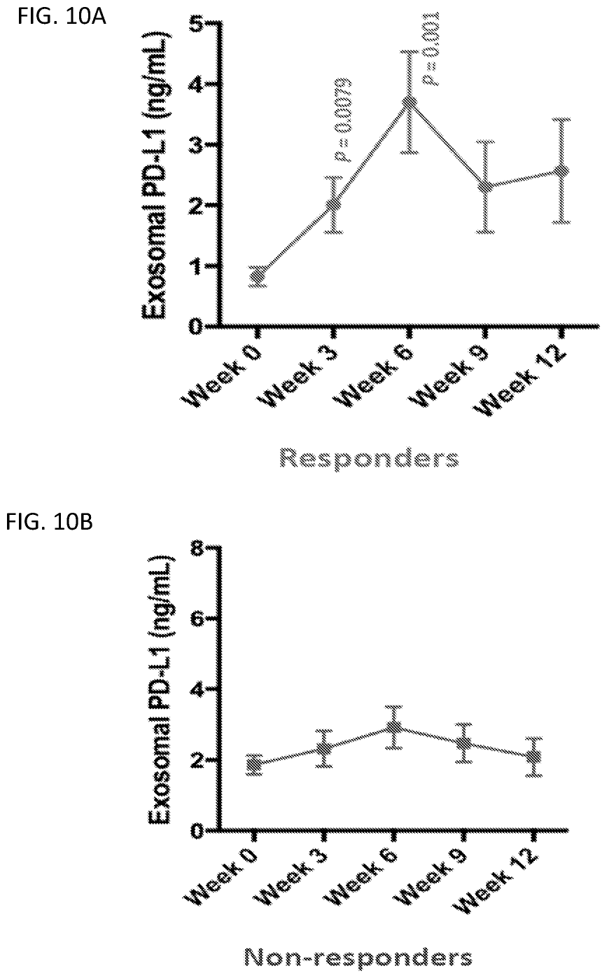

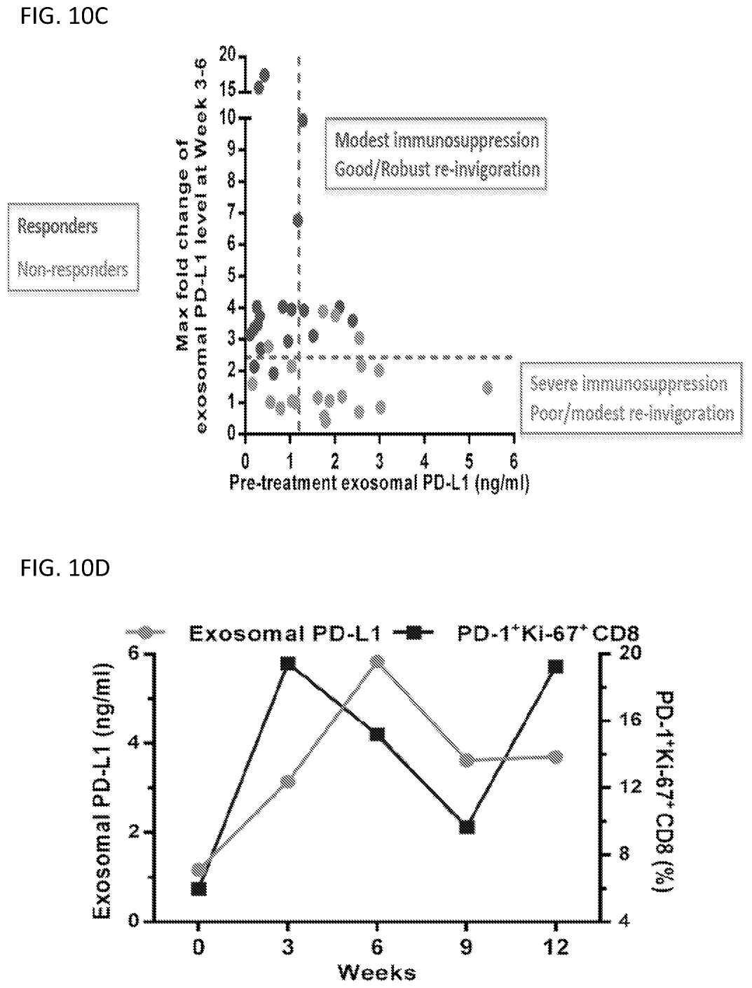

Subjects were administered a 12 week treatment of pembrolizumab, and exosomal PD-L1 levels were measured at weeks 0, 3, 6, 9, and 12 in patients who responded (“responders) and patients who failed to respond (“non-responders”) to treatment. FIGS. 10A and 10B depict these experimental results measuring exosomal PD-L1 levels in responders (FIG. 10A) and non-responders (FIG. 10B) across 12 weeks of pembrolizumab treatment, with week 0 being before the first treatment of pembrolizumab. Subjects who responded had significant fold increases in exosomal PD-L1 levels at weeks 3 and 6 of treatment compared to levels at week 0; whereas subjects who failed to respond to treatment also failed to have increases in exosomal PD-L1 levels compared to levels at week 0. FIG. 10C depicts the maximum fold change in exosomal PD-L1 levels at weeks 3 and 6 compared to the levels of exosomal PD-L1 prior to pembrolizumab treatment (“pre-treatment”). Subjects who responded had significant maximum-fold increases in exosomal PD-L1 levels at weeks 3 and 6 of treatment compared to levels at week 0 (y-axis); whereas subjects who failed to respond to treatment also failed to have maximum-fold increases in exosomal PD-L1 levels compared to levels at week 0. This appeared to be at least related to the levels of exosomal PD-L1 prior to treatment (x-axis). There was significant and even robust reinvigoration in individuals who responded to treatment with the largest maximum fold changes; whereas individual non-responders had severe immunosuppression correlating with a poor or modest reinvigoration of CD8 positive T cells. PD-1 positive, Ki-67 positive, CD8 positive T cell levels also precede the elevations in exosomal PD-L1 in responders (FIG. 10D), supporting the conclusion that there were robust CD8 positive T cells which in turn appeared to affect the exosomal PD-L1 levels and appeared to lead to the increases in the exosomal PD-L1 levels in responders at weeks 3 and 6 of treatment.

### Example 17

CD155 and PD-L1 were analyzed on myeloid cell-derived exosomes (CD11b+) (Melanoma) (FIGS. 11A-11D). Pre-treatment levels of CD155 and PD-L1 on myeloid cell-derived exosomes (captured with anti-CD11b antibody) are depicted in FIG. 11A. Patients with metastatic melanoma were treated with anti-PD-1 monotherapy (n=20 patients). Bar plots of RECIST response categories (CR/PR, SD/PD) by PD-L1 and CD155 levels on CD11b+ exosomes [CD155High (>0.3) vs. CD155Low (<0.3); PD-L1High (>8.0) vs PD-L1Low (<8.0)] in in treated patients is depicted in FIGS. 11B-11D. Results demonstrated that patients with high PD-L1 levels and low CD155 levels on myeloid cell-derived exosomes responded to anti-PD-1 therapy while patients with high PD-L1 level and high CD155 level were non-responders. These non-responders may be recommended for α-PD-1 and α-TIGIT combined therapy.

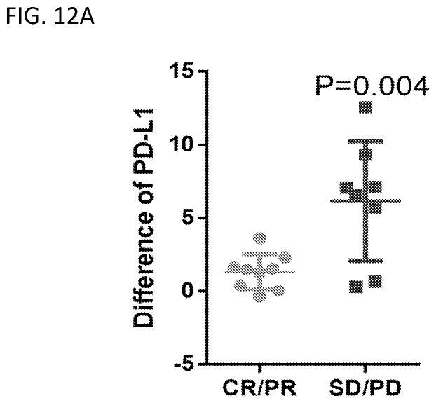

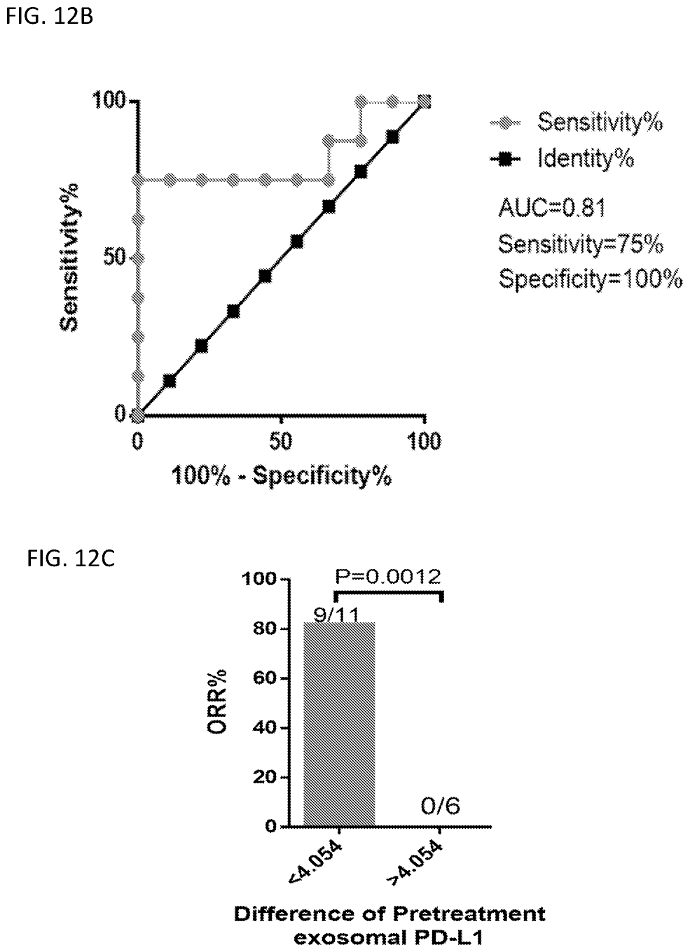

PD-L1 was analyzed on Tumor-associated Macrophage (TAM)-derived exosomes (CD163+) (FIGS. 12A-12C). The difference between baseline levels of PD-L1 on TAM-derived exosomes (CD163+) and total PD-L1 in melanoma patients treated with a-PD-1 therapy is shown in FIG. 12A. Difference (ng/ml)=[PD-L1(CD163+)]−[PD-L1(5H1)]. ROC curve analysis of the difference between pretreatment CD163+ exosomal PD-L1 and total exosomal PD-L1 in clinical responders compared to non-responders is shown in FIG. 12B. ORR for patients with high and low difference of exosomal PD-L1 (n=17 patients) is depicted in FIG. 12C. The difference of PD-L1 on CD163+ exosomes and total exosomal PD-L1 reflects the contribution of TAM to the exosomal PD-L1. Larger differences show higher levels of exosomal PD-L1 from TAMs. Patients with large a difference of pretreatment exosomal PD-L1 tend to be non-responders.

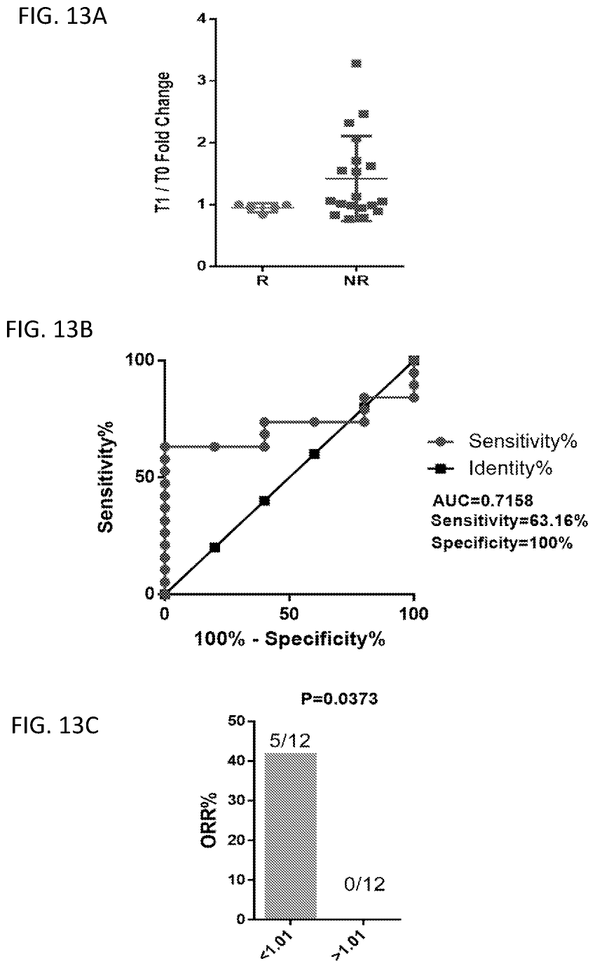

PD-L1 was analyzed on TAM-derived exosomes (Lung cancer) (FIGS. 13A-13C). FIG. 13A: Comparison of the fold change of PD-L1 on TAM-derived exosomes (CD163+) in patients with lung cancer treated with anti-PD-1 antibody. FIG. 13B: ROC curve analysis for the fold change of CD163+ exosomal PD-L1 in clinical responders compared to non-responders. FIG. 13C: ORR for patients with high and low fold change of CD163+ exosomal PD-L1 (n=24 patients). Results showed that lung cancer patients with an increase of TAM-derived exosomal PD-L1 after treatment did not response to a-PD-1 therapy.

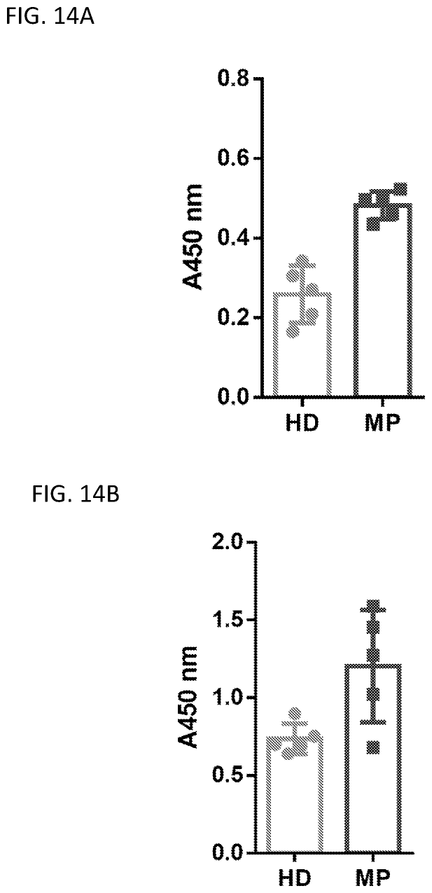

Co-expression of PD-L1 and CD155 was analyzed in melanoma patient exosomes (FIGS. 14A-14B). FIG. 14A: Comparison of the CD155 levels on PD-L1+ exosomes in melanoma patients compared to health donor. FIG. 14B: Comparison of the PD-L1 levels on exosomes captured by CD155 in melanoma patients compared to health donor. Patients whose exosomes co-express PD-L1 and CD155 may be suitable for anti-PD-1 and anti-TIGIT combination therapy. This was reflected in high levels of CD155 in PD-L1+ exosomes in patient plasma.

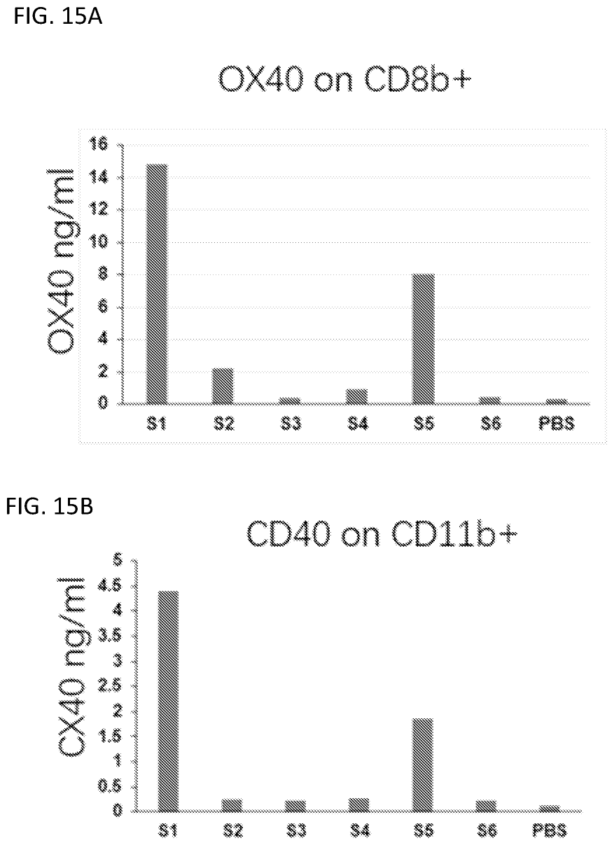

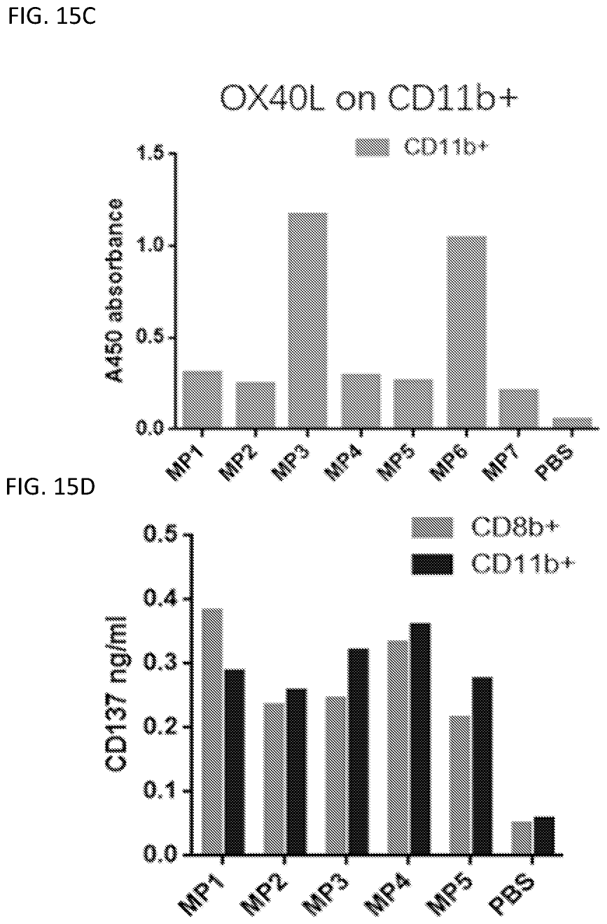

FIG. 15A shows detection of OX40 in CD8 T cell-derived exosomes (CD8b+ exosomes). FIG. 15B shows detection of CD40 on myeloid cell-derived exosomes (CD11b+ exosomes). FIG. 15C shows detection of OX40L on myeloid cell-derived exosomes (CD11b+ exosomes). FIG. 15D shows detection of CD137 on CD8 T cell-derived exosomes (CD8b+ exosomes) or myeloid cell-derived exosomes (CD11b+ exosome). Exosomal OX40 was detected on CD8 T cell-derived exosomes and exosomal OX40L. CD40 was detected on myeloid cell-derived exosomes. CD137 was detected on both CD8 T cell and myeloid cell-derived exosomes. These immune co-activation factors can be used to reflect the status of T cell activation.

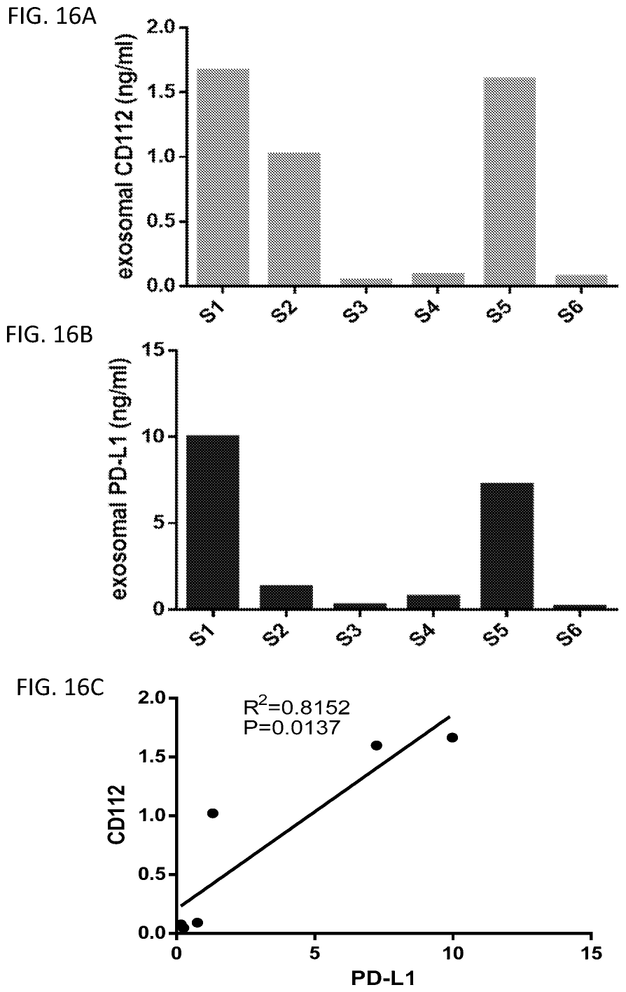

FIGS. 16A-16C depict CD112 on myeloid cells derived exosomes (CD11b+): correlative and non-correlative expression of CD112 and PD-L1. FIG. 16A: Detection of CD112 in myeloid cell-derived exosomes. FIG. 16B: Detection of total exosomal PD-L1. FIG. 16C: Pearson correlation of the CD11b+ exosomal CD112 and total exosomal PD-L1 in melanoma patients. CD112 was detected on myeloid cell-derived exosomes. In some patients, there is high exosomal CD112 and low exosomal PD-L1, suggesting these patients may suffer from CD112 mediated immunosuppression and do not response to α-PD-1 monotherapy.

### Other Embodiments

The recitation of a listing of elements in any definition of a variable herein includes definitions of that variable as any single element or combination (or subcoinbination) of listed elements. The recitation of an embodiment herein includes that embodiment as any single embodiment or in combination with any other embodiment or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for treating a patient who fails to respond to a checkpoint inhibitor therapy, the method comprising:
obtaining a first biological sample from the patient before the administration of the checkpoint inhibitor therapy, and a second biological sample from the patient after the patient has been administered at least one treatment of the checkpoint inhibitor therapy,
wherein each of the first and second biological samples comprises an extracellular vesicle comprising a source-specific marker and a checkpoint protein,
assessing the levels of the source-specific marker and checkpoint protein on the extracellular vesicles from the first and second biological samples,
wherein when the amount of the checkpoint protein from the second biological sample is elevated above the amount of the checkpoint protein from the first biological sample, an alternative treatment is administered to the patient.

2. The method of claim 1, wherein the checkpoint inhibitor therapy comprises an inhibitor of signaling of a first checkpoint protein.

3. The method of claim 2, wherein the alternative treatment comprises a second checkpoint inhibitor therapy comprising an inhibitor of signaling of a second checkpoint protein.

4. The method of claim 1, wherein the source-specific marker is selected from the group consisting of a marker for bone marrow derived antigen presenting cells, a marker for stroma cells, a marker for T cells, a marker for B cells, a marker for cancer cells, a marker for liver cells, a marker for stomach cells, a marker for pancreatic cells, a marker for small intestine cells, a marker for large intestine cells, a marker for endocrine cells, a marker for epithelial cells, a marker for endothelial cells, a marker for mesodermal cells, a marker for humoral cells, a marker for bone marrow, a marker for bone cells, a marker for nervous system cells, a marker for brain cells, a marker for neurons, a marker for glial cells, a marker for astrocytes, a marker for microglia, a marker for ependymal cells, a marker for stem cells, a marker for smooth muscle cells, a marker for cardiac cells, a marker for cardiac muscle cells, or a marker for basal cells.

5. The method of claim 4, wherein the marker for bone marrow derived antigen presenting cells comprises a marker for macrophages.

6. The method of claim 4, wherein the marker for macrophages is selected from the group consisting of a marker for M2 macrophages, a marker for M1 macrophages, a marker for tumor-associated macrophages, CD163, CD115, or CD11b.

7. The method of claim 4, wherein the bone marrow derived antigen presenting cells comprises M2 macrophages or tumor-associated macrophages.

8. The method of claim 4, wherein the marker for T cells comprises a marker for CD8+ T cells, a marker for CD4+ T cells, or a marker for regulatory T cells.

9. The method of claim 4, wherein the marker for stromal cells comprises CD44, CD90, CD105, or Fibroblast Surface Antigen (SFA).

10. The method of claim 2, wherein the first checkpoint inhibitor therapy comprises an inhibitor of programmed cell death protein 1 (PD-1) or programmed death-ligand protein 1 (PD-L1).

11. The method of claim 10, wherein the inhibitor of PD-L1 comprises atezolizumab.

12. The method of claim 1, wherein the source-specific marker is selected from the group consisting of CD63, CD9, C81, CD163, CD11b, CD11c, CD115, Gr-1, CD8, CD4, CD44, CD90, CD105, SFA, epidermal growth factor receptor (EGFR), vascular endothelial growth factor receptor (VEGFR), PD-L1, CD155, CD112, B7-1, B7-2, B7-H, Galectin-9, Siglec-15, OX40 receptor ligand (OX40L), major histocompatibility complex class II molecules (MHC-II), ICAM-1, LFA-1, B7-H2, CD40, CD70, CD48, CD52, CD137, TL1A, GITRL, CD30L, T cell immunoglobin and mucin domain 1 (TIM1), TIM3, TIM4, signaling lymphocytic activation molecule (SLAM), tumor necrosis factor superfamily member 14 (TNFSF14 or LIGHT), herpesvirus entry mediator (HVEM), PD-1, cytotoxic T lymphocyte-associated protein (CTLA-4), T cell immunoreceptor with Ig and immunoreceptor tyrosine-based inhibitory motif domains (TIGIT), Poliovirus Receptor Related Immunoglobulin Domain Containing (PVRIG), lymphocyte-activation gene 3 (LAG3), OX40, V-set and immunoglobulin domain containing 3 (VISG3), VISG8, inducible T cell costimulator (ICOS), CD28, CD40L, death receptor 3 (DR3), glucocorticoid-induced tumor necrosis factor-related protein (GITR), CD30, CD2, CD226, CD160, B and T lymphocyte attenuator (BTLA), programed death-1 homolog (PD-1H), leukocyte-associated immunoglobulin-like receptor 1 (LAIR1), or natural killer cell receptor 2B4 (NKCR2B4).

13. The method of claim 3, wherein the second checkpoint protein comprises: PD-L1, CD155, CD112, B7-1, B7-2, B7-H3, Galectin-9, Siglec-15, MHC-II, B7-H2, CD70, CD48, CD52, CD137, TL1A, GITRL, CD30L, TIM1, TIM3, TIM4, SLAM, LIGHT, HVEM, PD-1, CTLA-4, TIGIT, PVRIG, LAG3, VISG-3, VISG-8, ICOS, CD28, DR3, GITR, CD30, CD2, CD226, CD160, BTLA, PD1H, LAIR1, or NKCR2B4.

14. The method of claim 3, wherein the second checkpoint protein and the source-specific marker are the same.

15. The method of claim 3, wherein the second checkpoint protein and the source-specific marker differ.

16. The method of claim 3, wherein the second checkpoint protein is a receptor and the inhibitor of the signaling of the second checkpoint protein is an inhibitor of a ligand to the receptor.

17. The method of claim 3, wherein the second checkpoint protein is a ligand and the inhibitor of the signaling of the second checkpoint protein is an inhibitor of a receptor to the ligand.

18. The method of claim 3, wherein the second checkpoint protein is a ligand and a receptor.

19. The method of claim 3, wherein the second checkpoint protein is PD-1.

20. The method of claim 3, wherein the second checkpoint protein is CD155 and the inhibitor therapy is a TIGIT inhibitor.

21. The method of claim 1, wherein the difference in total amounts of extracellular vesicles between the first and second biological samples are normalized.

22. The method of claim 21, wherein the normalizing comprises measuring in each of the first and second biological samples: extracellular vesicles comprising CD9, extracellular vesicles comprising CD63, and extracellular vesicles comprising CD81, thereby measuring the total amount of extracellular vesicles in each of the first and second biological samples.

23. The method of claim 21, wherein in the normalizing, a total amount of the second checkpoint protein in the total amount of extracellular vesicles is obtained for each of the first and second biological samples.

24. The method of claim 23, wherein the administering is when the amount of the second checkpoint protein on the extracellular vesicle that has the source-specific marker in the second biological sample per the total amount of the second checkpoint protein in the total amount of extracellular vesicles in the second biological sample is elevated above the amount of the second checkpoint protein on the extracellular vesicle that has the source-specific marker of the first biological sample per the total amount of the second checkpoint protein in the total amount of extracellular vesicles in the first biological sample.

25. The method of claim 1, wherein the amount of the checkpoint protein on the extracellular vesicle that has the source-specific marker of each of the first and second biological samples is obtained by binding the checkpoint protein to a labelled antibody.

26. The method of claim 1, wherein the extracellular vesicle that has a source-specific marker is not from a cancer cell.

27. The method of claim 1, wherein the source-specific marker excludes a marker of a cancer cell.

28. The method of claim 1, wherein the alternative treatment is administered when the amount of the checkpoint protein from the second biological sample is elevated at least 1.5, 2, 3, 4, 5, 6, 7, 8, 9, 10, or 20 fold above the amount of the checkpoint protein from the first biological sample.

29. A method of identifying whether a subject is responsive to a checkpoint protein inhibitor therapy, the method comprising:
i) detecting the level of an extracellular vesicle-bound immune regulatory protein obtained from the subject;
ii) detecting the level of a marker for the extracellular vesicle; and
iii) identifying the subject as non-responsive to treatment wherein:
a) the level of the extracellular vesicle-bound immune regulatory protein and the level of the marker for the extracellular vesicle correlate in a population of individuals, and
b) there is a X increase in the level of the extracellular vesicle-bound immune regulatory protein from the subject per the level of marker for the extracellular vesicle from the subject above Y,
wherein Y equals the mean of Z and X is the standard deviation of Z,
wherein Z is levels of the extracellular vesicle-bound immune regulatory protein of each individual from the population of individuals divided by levels of the marker for the extracellular vesicle of said each individual from the population of individuals,
wherein X is at least 0.68.

30. The method of claim 29, wherein the marker for the extracellular vesicle comprises: CD63, CD9, C81, CD163, CD11b, CD11c, CD115, Gr-1, CD8, CD4, CD44, CD90, CD105, SFA, epidermal growth factor receptor (EGFR), vascular endothelial growth factor receptor (VEGFR), PD-L1, CD155, CD112, B7-1, B7-2, B7-H, Galectin-9, Siglec-15, OX40 receptor ligand (OX40L), major histocompatibility complex class II molecules (MHC-II), ICAM-1, LFA-1, B7-H2, CD40, CD70, CD48, CD52, CD137, TL1A, GITRL, CD30L, T cell immunoglobin and mucin domain 1 (TIM1), TIM3, TIM4, signaling lymphocytic activation molecule (SLAM), tumor necrosis factor superfamily member 14 (TNFSF14 or LIGHT), herpesvirus entry mediator (HVEM), PD-1, cytotoxic T lymphocyte-associated protein (CTLA-4), T cell immunoreceptor with Ig and immunoreceptor tyrosine-based inhibitory motif domains (TIGIT), Poliovirus Receptor Related Immunoglobulin Domain Containing (PVRIG), lymphocyte-activation gene 3 (LAG3), OX40, V-set and immunoglobulin domain containing 3 (VISG3), VISG8, inducible T cell costimulator (ICOS), CD28, CD40L, death receptor 3 (DR3), glucocorticoid-induced tumor necrosis factor-related protein (GITR), CD30, CD2, CD226, CD160, B and T lymphocyte attenuator (BTLA), programed death-1 homolog (PD-1H), leukocyte-associated immunoglobulin-like receptor 1 (LAIR1), or natural killer cell receptor 2B4 (NKCR2B4).

31. The method of claim 29, wherein the extracellular vesicle-bound immune regulatory protein comprises: PD-L1, CD155, CD112, B7-1, B7-2, B7-H3, Galectin-9, Siglec-15, OX40L, MHC-II, B7-H2, CD40, CD70, CD48, CD52, CD137, TL1A, GITRL, CD30L, TIM1, TIM3, TIM4, SLAM, LIGHT, HVEM, PD-1, CTLA-4, TIGIT, PVRIG, LAG3, OX40, VISG-3, VISG-8, ICOS, CD28, CD40L, DR3, GITR, CD30, CD2, CD226, CD160, BTLA, PD1H, LAIR1, or NKCR2B4.

32. The method of claim 29, wherein X is at least 1, at least 1.5, at least 2, at least 2.5, or at least 3.

33. A method of increasing the efficacy of an anti-cancer medication in a patient in need thereof, the method comprising: contacting a biological sample from the patient with a first reagent,
wherein the biological sample comprises a cancer-promoting extracellular vesicle comprising a checkpoint protein and a source-specific marker, the checkpoint protein suppressing an immune response to the cancer, wherein in the contacting, the first reagent binds the cancer-promoting extracellular vesicles, thereby removing the cancer-promoting extracellular vesicles from the biological sample and obtaining a purified biological sample; and introducing the purified biological sample to the patient thereby increasing the efficacy of the anti-cancer medication.

34. The method of claim 33, wherein the first reagent binds the source-specific marker, thereby removing the cancer-promoting extracellular vesicles from the biological sample.

35. The method of claim 33, wherein the anti-cancer medication comprises a checkpoint inhibitor therapy comprising an inhibitor of signaling of the checkpoint protein.

36. The method of claim 33, wherein the source-specific marker is selected from the group consisting of a marker for bone marrow derived antigen presenting cells, a marker for stroma cells, a marker for T cells, a marker for B cells, a marker for cancer cells, a marker for liver cells, a marker for stomach cells, a marker for pancreatic cells, a marker for small intestine cells, a marker for large intestine cells, a marker for endocrine cells, a marker for epithelial cells, a marker for endothelial cells, a marker for mesodermal cells, a marker for humoral cells, a marker for bone marrow, a marker for bone cells, a marker for nervous system cells, a marker for brain cells, a marker for neurons, a marker for glial cells, a marker for astrocytes, a marker for microglia, a marker for ependymal cells, a marker for stem cells, a marker for smooth muscle cells, a marker for cardiac cells, a marker for cardiac muscle cells, or a marker for basal cells.

37. The method of claim 36, wherein the marker for bone derived antigen presenting cells comprises a marker for macrophages.

38. The method of claim 36, wherein the marker for macrophages comprises a marker for M2 macrophages, M1 macrophages, or tumor-associated macrophages.

39. The method of claim 36, wherein the marker for macrophages comprises CD63, CD115, or CD11b.

40. The method of claim 36, wherein the bone marrow derived antigen presenting cells comprises M2 macrophages or tumor-associated macrophages.

41. The method of claim 36, wherein the marker for T cells comprise a marker for CD8+ T cells, a marker for CD4+ T cells, or a marker for regulatory T cells.

42. The method of claim 36, wherein the marker for stromal cells comprises CD44, CD90, CD105, or Fibroblast Surface Antigen (SFA).

43. The method of claim 35, wherein the checkpoint inhibitor therapy comprises an inhibitor of programmed cell death protein 1 (PD-1) or an inhibitor of programmed death-ligand protein 1 (PD-L1).

44. The method of claim 43, wherein the inhibitor of PD-L1 comprises atezolizumab.

45. The method of claim 33, wherein the source-specific marker comprises: CD63, CD9, C81, CD163, CD11b, CD11c, CD115, Gr-1, CD8, CD4, CD44, CD90, CD105, SFA, epidermal growth factor receptor (EGFR), vascular endothelial growth factor receptor (VEGFR), PD-L1, CD155, CD112, B7-1, B7-2, B7-H, Galectin-9, Siglec-15, OX40 receptor ligand (OX40L), major histocompatibility complex class II molecules (MHC-II), ICAM-1, LFA-1, B7-H2, CD40, CD70, CD48, CD52, CD137, TL1A, GITRL, CD30L, T cell immunoglobin and mucin domain 1 (TIM1), TIM3, TIM4, signaling lymphocytic activation molecule (SLAM), tumor necrosis factor superfamily member 14 (TNFSF14 or LIGHT), herpesvirus entry mediator (HVEM), PD-1, cytotoxic T lymphocyte-associated protein (CTLA-4), T cell immunoreceptor with Ig and immunoreceptor tyrosine-based inhibitory motif domains (TIGIT), Poliovirus Receptor Related Immunoglobulin Domain Containing (PVRIG), lymphocyte-activation gene 3 (LAG3), OX40, V-set and immunoglobulin domain containing 3 (VISG3), VISG8, inducible T cell costimulator (ICOS), CD28, CD40L, death receptor 3 (DR3), glucocorticoid-induced tumor necrosis factor-related protein (GITR), CD30, CD2, CD226, CD160, B and T lymphocyte attenuator (BTLA), programed death-1 homolog (PD-1H), leukocyte-associated immunoglobulin-like receptor 1 (LAIR1), or natural killer cell receptor 2B4 (NKCR2B4).

46. The method of claim 33, wherein the checkpoint protein comprises: PD-L1, CD155, CD112, B7-1, B7-2, B7-H3, Galectin-9, Siglec-15, B7-H2, CD70, CD48, CD52, CD137, TL1A, GITRL, CD30L, TIM1, TIM3, TIM4, SLAM, LIGHT, HVEM, PD-1, CTLA-4, TIGIT, PVRIG, LAG3, OX40, VISG-3, VISG-8, ICOS, CD28, DR3, GITR, CD30, CD2, CD226, CD160, BTLA, PD1H, LAIR1, or NKCR2B4.

47. The method of claim 33, wherein the checkpoint protein and the source-specific marker are the same.

48. The method of claim 33, wherein the checkpoint protein and the source-specific marker differ.

49. A method for treating cancer in a patient in need thereof, the method comprising:
assessing the levels of PD-L1 and/or PD-1 and the levels of CD155 and/or CD112 and/or TIGIT in a biological sample comprising an extracellular vesicle obtained from the patient before administration of a first therapy,
wherein when the the level of PD-L1 and/or PD-1 is high and the level of CD155 and/or CD112 and/or TIGIT is low in comparison to a reference sample, the first therapy is administered to the patient, and
wherein when the levels of PD-L1 and/or PD-1 and the levels of CD155 and/or CD112 and/or TIGIT are high in comparison to a reference sample, a second therapy is administered alone or together with the first therapy to the patient.

50. The method of claim 49, wherein the first therapy comprises an inhibitor of PD-1 or PD-L1 and the second therapy comprises an inhibitor of TGIT.

51. The method of claim 50, wherein the inhibitor is selected from the group consisting of an antibody, a chemical compound, or an siRNA.

52. A method for treating cancer in a patient in need thereof, the method comprising:
assessing the level of CD155 and/or CD112 in a biological sample comprising an extracellular vesicle obtained from the patient before administration of a first therapy, and assessing the level of CD155 and/or CD112 in a biological sample comprising an extracellular vesicle obtained from the patient after administration of a first therapy,
wherein when the the level of CD155 and/or CD112 in the second biological sample is increased in comparison to the first biological sample, the patient is determined to not be responding to the first therapy, and a second therapy is administered to the patient.

53. The method of claim 52, wherein the first therapy comprises an inhibitor of PD-1 or PD-L1 and the second therapy comprises an inhibitor of TGIT.

54. The method of claim 53, wherein the inhibitor is selected from the group consisting of an antibody, a chemical compound, or an siRNA.

55. A method for treating cancer in a patient in need thereof, the method comprising:
assessing the level of a co-stimulatory factor in a first biological sample comprising an extracellular vesicle obtained from the patient before administration of a therapy, and
assessing the level of the co-stimulatory factor in a second biological sample comprising an extracellular vesicle obtained from the patient after administration of a therapy,
wherein when the level of the co-stimulatory factor is increased in the second biological sample in comparison to the first biological sample, the patient is determined to be responding to therapy, and the therapy is continued.

56. The method of claim 55, further comprising assessing the level of the co-stimulatory factor in a third biological sample comprising an extracellular vesicle obtained from the patient,
wherein when the level of the co-stimulatory factor is decreased in the third biological sample in comparison to the first biological sample, the patient is determined to be responding to therapy, and the therapy is continued, and
wherein when the level of the co-stimulatory factor is increased in the third biological sample in comparison to the first biological sample, the patient is determined to not be responding to therapy, and the therapy is discontinued and a different therapy is administered to the patient.

57. The method of claim 55, wherein the second biological sample is obtained three weeks after the first biological sample.

58. The method of claim 56, wherein the third biological sample is obtained 2, 3, 4, 5, 6, 7, 8, or 9 weeks after the second biological sample.

59. The method of claim 52, wherein the co-stimulatory factor is selected from the group consisting of CD40, CD40L, OX40, OX40L, CD137, and CD137L.

60. A method for treating cancer in a patient in need thereof, the method comprising:
assessing the level of phosphorylated HRS in a biological sample comprising tumor tissues or an extracellular vesicle obtained from the patient,
wherein when the level of phosphorylated HRS is high in comparison to a reference sample, the patient is administered a combination therapy.

61. The method of claim 60, wherein the combination therapy comprises a drug that inhibits PD-1 or PD-L1 and a drug that inhibits the MAPK pathway.

62. The method of claim 49, wherein the cancer is melanoma.
